SEQUENCE LISTING (28 sequences)

SEQ ID NO: 1 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 3 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 1017 bp, synthetic construct
  source 1..1017: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 5 - DNA, 1572 bp, synthetic construct
  source 1..1572: mol_type = other DNA
gctggcctgtggctgggcctggtgtggcagaagctgttgctgtggggggctgcctctgccctgagcctggctggggctagcctggtgttgagcctgctgcagagagtggctagctatgctagaaagtggcagcagatgagacccatccccacagtggcaagagcctaccctctggtgggccatgccctgctgatgaagcctgatggcagagagttctttcagcagatcatagagtacacagaggagtacagacacatgcccctgctgaaactgtgggtgggccctgtgcccatggtggctctgtacaatgctgagaatgtggaggtgattctgacaagcagcaagcagattgacaagagctccatgtacaagttcctggagccctggctgggcctgggactgctgacaagcactggcaacaagtggagaagcagaagaaagatgctgacccccaccttccacttcaccatcctggaggacttcctggacattatgaatgagcaagccaacatcctggtgaaaaagctggaaaagcacatcaaccaagaggccttcaactgcttcttctacatcaccctgtgtgccctggacatcatctgtgagacagccatgggcaagaacatcggggcccagagcaatgatgactctgagtatgtgagagctgtgtacagaatgtctgagatgatcttcagaagaatcaagatgccctggctgtggctggacctgtggtacctgatgttcaaggagggctgggagcacaaaaagagcctgcagatcctgcacaccttcaccaactctgtgatcgccgagagagccaatgagatgaatgccaatgaggactgcagaggggatggcagaggctctgcccctagcaagaacaagagaagagccttcctggacctgttgctgtctgtgaccgacgatgagggcaacagactgagccatgaggacatcagagaagaggtggacacatttatgtttgagggccatgacaccacagctgctgccataaactggagcctgtacctgctgggcagcaaccctgaggtgcagaagaaggtggaccatgagctggatgatgtgtttggcaagtctgacagacctgccacagtggaggacctgaagaagctgagatacctggagtgtgtgatcaaggagaccctgagactgttcccctctgtgcccctgtttgctagatctgtgtctgaggactgtgaggtggctggctatagagtgctgaagggcacagaggctgtgatcatcccctatgccctgcacagagaccctagatacttccccaaccctgaggagtttcagcctgagagattcttccctgagaatgcccaaggcagacacccctatgcctatgtgccattctctgctggcccaagaaactgcattgggcagaagtttgctgtgatggaggagaagaccatcctgagctgcatcttgagacacttctggattgagagcaatcagaagagagaggagctgggcctggaggggcagctgatactgagaccaagcaatggcatctggatcaagctgaagagaagaaatgctgatgagaga

SEQ ID NO: 6 - DNA, 1572 bp, synthetic construct
  source 1..1572: mol_type = other DNA
gctggcctgtggctgggcctggtgtggcagaagctgctgctgtggggagctgcctctgccctgtctctggctggagccagcctggtgctgagcctgctgcagagagtggccagctatgccaggaaatggcaacagatgagacctatccccacagtggccagagcctaccccctggttggccatgccctgctgatgaagcctgatggcagagagttcttccagcagatcattgagtacacagaagagtacagacacatgcctctgctgaagctgtgggtgggccctgtgcccatggtggccctgtacaatgcagagaatgtggaagtgatcctgaccagcagcaagcagatagacaagtccagcatgtacaagttcctggaaccttggctgggcctgggcctgctcacctccacaggcaacaagtggagaagcagaaggaagatgctgaccccaaccttccacttcaccatcctggaggactttctggacatcatgaatgagcaggccaacatcctggtcaaaaaactggaaaagcacatcaaccaggaagccttcaactgcttcttctacatcaccctgtgtgccctggacatcatctgcgagacagccatgggcaagaacatcggagcccagagcaatgatgactctgaatatgtcagggcagtgtacagaatgtctgagatgatcttccggcggatcaagatgccttggctgtggctggacctgtggtacctgatgttcaaagagggctgggagcacaagaagagcctgcagatcctgcacaccttcaccaacagcgtgattgctgagcgggccaatgaaatgaacgccaatgaggactgcagaggagatggcagaggctctgcccccagcaagaacaagagaagagccttcctggacctgctgctgtccgtgacagatgatgagggcaacagactgagccacgaggacatcagagaggaagtggacacctttatgtttgaaggccatgacaccacagctgctgccatcaactggagcctgtacctcctgggcagcaaccctgaggtgcagaagaaggtggaccatgagctggatgatgtgtttggcaagtctgacagacctgccacagtggaagacctgaagaaactcaggtacctggaatgtgtgatcaaagagaccctgagactgttcccaagtgtgcctctgtttgccagatctgtctctgaggactgtgaagtggctggctacagagtgctgaagggcacagaggcagttatcatcccctatgccctgcacagagaccccagatacttccccaaccctgaagagttccagcctgagagattcttcccagagaatgcccagggcagacacccttatgcctatgtgcccttctcagctggacctagaaactgcataggccaaaagtttgcagtgatggaagagaagaccatcctgagctgcatcctgaggcacttctggattgagagcaaccagaagagagaggaactgggcctggaaggacagctgatcctgaggcctagcaatggcatctggatcaagctgaaaagaagaaatgctgatgagagg

SEQ ID NO: 7 - DNA, 1572 bp, synthetic construct
  source 1..1572: mol_type = other DNA
gctggcctgtggctgggcctggtgtggcagaaactgctgctgtggggagctgcctctgccctgtctctggctggagcctcccttgtcctgtccctcctgcaaagagttgccagctatgccagaaagtggcagcagatgagacccatccccacagtggccagggcctacccactggtgggccatgccctgctgatgaagcctgatggcagagaattctttcagcagatcatagagtacacagaggagtacagacacatgcctctgctgaagctgtgggtgggccctgtgcctatggtggccctgtacaatgctgagaatgtggaagtgatcctgaccagcagcaagcagattgacaagagcagcatgtacaagttcctggagccttggctgggcctgggcctgctgaccagcacaggcaacaagtggagaagcaggagaaagatgctgacccccaccttccacttcaccatcctggaagacttcctggacatcatgaatgagcaggccaacatcctggtgaaaaagctggaaaaacacatcaaccaggaagccttcaactgcttcttctacattaccctgtgtgccctggacatcatctgcgagacagccatgggaaaaaacatcggagctcagagcaatgatgacagcgagtatgtgagagcagtgtaccggatgagcgaaatgatcttcagacggatcaagatgccctggctgtggctggacctgtggtacctcatgtttaaggagggctgggaacacaagaagagcctgcagatcctgcacaccttcacaaacagtgtgatcgctgaaagggccaacgagatgaatgccaatgaggattgcagaggcgatggccgcggctccgcccctagcaagaacaagagaagagccttcctggacctgctgctgtctgtcaccgatgacgagggcaaccggctgtctcatgaggacatcagagaagaggtggacaccttcatgtttgagggccacgacaccacagccgccgccatcaactggagcctgtacctgcttggcagcaaccctgaggtgcaaaagaaggtggaccatgagctggatgatgtttttggcaaatctgacagacctgccacagtggaggacctgaagaaactgagatacctggagtgtgtgatcaaggaaaccctgagactcttccctagtgtgcctctgtttgccagatctgtctcagaggactgtgaggtggctggctacagagtgctgaagggcacagaagcagtgatcatcccctatgccctgcacagagaccccagatacttccccaaccctgaggaattccagccagagaggttcttccctgagaatgcccagggaagacacccatatgcctatgtgcctttctctgctggccccagaaactgcattggacagaagtttgctgtgatggaggaaaagaccatcctgagctgcatcctgagacacttctggatagaaagcaaccagaagagggaagagctgggcctggaaggccagctgatcctgaggcctagcaatggcatctggatcaaactgaagagaagaaatgcagatgagaga

SEQ ID NO: 8 - DNA, 1572 bp, synthetic construct
  source 1..1572: mol_type = other DNA
gctggcctgtggctgggcctggtgtggcagaagctgctgctgtggggagctgcctctgccctgtccctggctggagcctctctggtgctgagcctgctgcagagagtggccagctatgccaggaaatggcaacagatgagacccatcccaacagtggccagagcctaccccctggttggccatgccctgctgatgaagcctgatggcagagagttcttccagcagatcattgagtacacagaggaatacagacacatgcctctgctgaagctgtgggtgggccctgtgcccatggtggccctgtacaatgcagagaatgtggaagtgatcctgacctccagcaagcagatagacaagagcagcatgtacaagttcctggaaccttggctgggccttggcctgctcaccagcacaggcaacaagtggagaagcagaaggaagatgctgacccctaccttccacttcaccatcctggaggactttctggacatcatgaatgagcaggccaacatcctggtcaaaaagctggaaaagcacatcaaccaggaggccttcaactgcttcttctacatcaccctgtgtgccctggacatcatctgcgagacagccatgggcaaaaacatcggagcccagagcaatgatgactctgagtatgtgcgggctgtgtaccggatgagcgaaatgatcttcagacggatcaagatgccctggctgtggctggacctctggtacctgatgttcaaagaaggctgggagcacaagaagagcctgcagatcctgcacaccttcaccaacagcgtgattgcagaaagagccaatgagatgaatgccaacgaggactgcagaggagatggcagaggctctgcccctagcaagaacaagagaagggcctttctggacctgctgctgtctgtcacagatgatgagggcaacagactgagccacgaggacatcagagaggaagtggacaccttcatgtttgaaggccatgacaccacagctgctgccatcaactggtccctgtacctgctgggcagcaaccctgaggtgcagaagaaggtggaccatgagctggatgatgtgtttggcaagtctgacagacctgccacagtggaagacctgaaaaagctgagatacctggaatgtgtgatcaaagagaccctgagactgttcccttctgtgcctctgtttgccagaagtgtttctgaggactgtgaagtggctggctacagagtgctgaagggcacagaggcagtcatcatcccctatgccctgcacagagaccccaggtacttccccaaccctgaggagttccagcctgaaagattcttcccagagaatgcccagggcagacacccatatgcctatgtgcccttctcagctggacctagaaactgcataggacaaaagtttgcagtgatggaagagaagaccatcctgagctgcatcctgaggcacttctggattgagagcaaccagaaaagagaggaactgggcctggaaggccagctgatcctcaggcctagcaatggcatctggatcaagctgaagagaaggaatgctgatgagaga

SEQ ID NO: 9 - DNA, 1572 bp, synthetic construct
  source 1..1572: mol_type = other DNA
gcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgc

SEQ ID NO: 10 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 11 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 12 - DNA, 3961 bp, synthetic construct
  source 1..3961: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctcgaggagcttggcccattgcatacgttgtatccatatcataatatgtacatttatattggctcatgtccaacattaccgccatgttactagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcgcgccgccaccatggctggcctgtggctgggcctggtgtggcagaagctgctgctgtggggagctgcctctgccctgtccctggctggagcctctctggtgctgagcctgctgcagagagtggccagctatgccaggaaatggcaacagatgagacccatcccaacagtggccagagcctaccccctggttggccatgccctgctgatgaagcctgatggcagagagttcttccagcagatcattgagtacacagaggaatacagacacatgcctctgctgaagctgtgggtgggccctgtgcccatggtggccctgtacaatgcagagaatgtggaagtgatcctgacctccagcaagcagatagacaagagcagcatgtacaagttcctggaaccttggctgggccttggcctgctcaccagcacaggcaacaagtggagaagcagaaggaagatgctgacccctaccttccacttcaccatcctggaggactttctggacatcatgaatgagcaggccaacatcctggtcaaaaagctggaaaagcacatcaaccaggaggccttcaactgcttcttctacatcaccctgtgtgccctggacatcatctgcgagacagccatgggcaaaaacatcggagcccagagcaatgatgactctgagtatgtgcgggctgtgtaccggatgagcgaaatgatcttcagacggatcaagatgccctggctgtggctggacctctggtacctgatgttcaaagaaggctgggagcacaagaagagcctgcagatcctgcacaccttcaccaacagcgtgattgcagaaagagccaatgagatgaatgccaacgaggactgcagaggagatggcagaggctctgcccctagcaagaacaagagaagggcctttctggacctgctgctgtctgtcacagatgatgagggcaacagactgagccacgaggacatcagagaggaagtggacaccttcatgtttgaaggccatgacaccacagctgctgccatcaactggtccctgtacctgctgggcagcaaccctgaggtgcagaagaaggtggaccatgagctggatgatgtgtttggcaagtctgacagacctgccacagtggaagacctgaaaaagctgagatacctggaatgtgtgatcaaagagaccctgagactgttcccttctgtgcctctgtttgccagaagtgtttctgaggactgtgaagtggctggctacagagtgctgaagggcacagaggcagtcatcatcccctatgccctgcacagagaccccaggtacttccccaaccctgaggagttccagcctgaaagattcttcccagagaatgcccagggcagacacccatatgcctatgtgcccttctcagctggacctagaaactgcataggacaaaagtttgcagtgatggaagagaagaccatcctgagctgcatcctgaggcacttctggattgagagcaaccagaaaagagaggaactgggcctggaaggccagctgatcctcaggcctagcaatggcatctggatcaagctgaagagaaggaatgctgatgagagataaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcccctaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 3961 bp, synthetic construct
  source 1..3961: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctcgaggagcttggcccattgcatacgttgtatccatatcataatatgtacatttatattggctcatgtccaacattaccgccatgttactagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcgcgccgccaccatggctggcctgtggctgggcctggtgtggcagaagctgctgctgtggggagctgcctctgccctgtctctggctggagccagcctggtgctgagcctgctgcagagagtggccagctatgccaggaaatggcaacagatgagacctatccccacagtggccagagcctaccccctggttggccatgccctgctgatgaagcctgatggcagagagttcttccagcagatcattgagtacacagaagagtacagacacatgcctctgctgaagctgtgggtgggccctgtgcccatggtggccctgtacaatgcagagaatgtggaagtgatcctgaccagcagcaagcagatagacaagtccagcatgtacaagttcctggaaccttggctgggcctgggcctgctcacctccacaggcaacaagtggagaagcagaaggaagatgctgaccccaaccttccacttcaccatcctggaggactttctggacatcatgaatgagcaggccaacatcctggtcaaaaaactggaaaagcacatcaaccaggaagccttcaactgcttcttctacatcaccctgtgtgccctggacatcatctgcgagacagccatgggcaagaacatcggagcccagagcaatgatgactctgaatatgtcagggcagtgtacagaatgtctgagatgatcttccggcggatcaagatgccttggctgtggctggacctgtggtacctgatgttcaaagagggctgggagcacaagaagagcctgcagatcctgcacaccttcaccaacagcgtgattgctgagcgggccaatgaaatgaacgccaatgaggactgcagaggagatggcagaggctctgcccccagcaagaacaagagaagagccttcctggacctgctgctgtccgtgacagatgatgagggcaacagactgagccacgaggacatcagagaggaagtggacacctttatgtttgaaggccatgacaccacagctgctgccatcaactggagcctgtacctcctgggcagcaaccctgaggtgcagaagaaggtggaccatgagctggatgatgtgtttggcaagtctgacagacctgccacagtggaagacctgaagaaactcaggtacctggaatgtgtgatcaaagagaccctgagactgttcccaagtgtgcctctgtttgccagatctgtctctgaggactgtgaagtggctggctacagagtgctgaagggcacagaggcagttatcatcccctatgccctgcacagagaccccagatacttccccaaccctgaagagttccagcctgagagattcttcccagagaatgcccagggcagacacccttatgcctatgtgcccttctcagctggacctagaaactgcataggccaaaagtttgcagtgatggaagagaagaccatcctgagctgcatcctgaggcacttctggattgagagcaaccagaagagagaggaactgggcctggaaggacagctgatcctgaggcctagcaatggcatctggatcaagctgaaaagaagaaatgctgatgagaggtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcccctaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 14 - DNA, 3961 bp, synthetic construct
  source 1..3961: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctcgaggagcttggcccattgcatacgttgtatccatatcataatatgtacatttatattggctcatgtccaacattaccgccatgttactagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcgcgccgccaccatggctggcctgtggctgggcctggtgtggcagaaactgctgctgtggggagctgcctctgccctgtctctggctggagcctcccttgtcctgtccctcctgcaaagagttgccagctatgccagaaagtggcagcagatgagacccatccccacagtggccagggcctacccactggtgggccatgccctgctgatgaagcctgatggcagagaattctttcagcagatcatagagtacacagaggagtacagacacatgcctctgctgaagctgtgggtgggccctgtgcctatggtggccctgtacaatgctgagaatgtggaagtgatcctgaccagcagcaagcagattgacaagagcagcatgtacaagttcctggagccttggctgggcctgggcctgctgaccagcacaggcaacaagtggagaagcaggagaaagatgctgacccccaccttccacttcaccatcctggaagacttcctggacatcatgaatgagcaggccaacatcctggtgaaaaagctggaaaaacacatcaaccaggaagccttcaactgcttcttctacattaccctgtgtgccctggacatcatctgcgagacagccatgggaaaaaacatcggagctcagagcaatgatgacagcgagtatgtgagagcagtgtaccggatgagcgaaatgatcttcagacggatcaagatgccctggctgtggctggacctgtggtacctcatgtttaaggagggctgggaacacaagaagagcctgcagatcctgcacaccttcacaaacagtgtgatcgctgaaagggccaacgagatgaatgccaatgaggattgcagaggcgatggccgcggctccgcccctagcaagaacaagagaagagccttcctggacctgctgctgtctgtcaccgatgacgagggcaaccggctgtctcatgaggacatcagagaagaggtggacaccttcatgtttgagggccacgacaccacagccgccgccatcaactggagcctgtacctgcttggcagcaaccctgaggtgcaaaagaaggtggaccatgagctggatgatgtttttggcaaatctgacagacctgccacagtggaggacctgaagaaactgagatacctggagtgtgtgatcaaggaaaccctgagactcttccctagtgtgcctctgtttgccagatctgtctcagaggactgtgaggtggctggctacagagtgctgaagggcacagaagcagtgatcatcccctatgccctgcacagagaccccagatacttccccaaccctgaggaattccagccagagaggttcttccctgagaatgcccagggaagacacccatatgcctatgtgcctttctctgctggccccagaaactgcattggacagaagtttgctgtgatggaggaaaagaccatcctgagctgcatcctgagacacttctggatagaaagcaaccagaagagggaagagctgggcctggaaggccagctgatcctgaggcctagcaatggcatctggatcaaactgaagagaagaaatgcagatgagagataaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcccctaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 15 - DNA, 3961 bp, synthetic construct
  source 1..3961: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctcgaggagcttggcccattgcatacgttgtatccatatcataatatgtacatttatattggctcatgtccaacattaccgccatgttactagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcgcgccgccaccatggctggcctgtggctgggcctggtgtggcagaagctgttgctgtggggggctgcctctgccctgagcctggctggggctagcctggtgttgagcctgctgcagagagtggctagctatgctagaaagtggcagcagatgagacccatccccacagtggcaagagcctaccctctggtgggccatgccctgctgatgaagcctgatggcagagagttctttcagcagatcatagagtacacagaggagtacagacacatgcccctgctgaaactgtgggtgggccctgtgcccatggtggctctgtacaatgctgagaatgtggaggtgattctgacaagcagcaagcagattgacaagagctccatgtacaagttcctggagccctggctgggcctgggactgctgacaagcactggcaacaagtggagaagcagaagaaagatgctgacccccaccttccacttcaccatcctggaggacttcctggacattatgaatgagcaagccaacatcctggtgaaaaagctggaaaagcacatcaaccaagaggccttcaactgcttcttctacatcaccctgtgtgccctggacatcatctgtgagacagccatgggcaagaacatcggggcccagagcaatgatgactctgagtatgtgagagctgtgtacagaatgtctgagatgatcttcagaagaatcaagatgccctggctgtggctggacctgtggtacctgatgttcaaggagggctgggagcacaaaaagagcctgcagatcctgcacaccttcaccaactctgtgatcgccgagagagccaatgagatgaatgccaatgaggactgcagaggggatggcagaggctctgcccctagcaagaacaagagaagagccttcctggacctgttgctgtctgtgaccgacgatgagggcaacagactgagccatgaggacatcagagaagaggtggacacatttatgtttgagggccatgacaccacagctgctgccataaactggagcctgtacctgctgggcagcaaccctgaggtgcagaagaaggtggaccatgagctggatgatgtgtttggcaagtctgacagacctgccacagtggaggacctgaagaagctgagatacctggagtgtgtgatcaaggagaccctgagactgttcccctctgtgcccctgtttgctagatctgtgtctgaggactgtgaggtggctggctatagagtgctgaagggcacagaggctgtgatcatcccctatgccctgcacagagaccctagatacttccccaaccctgaggagtttcagcctgagagattcttccctgagaatgcccaaggcagacacccctatgcctatgtgccattctctgctggcccaagaaactgcattgggcagaagtttgctgtgatggaggagaagaccatcctgagctgcatcttgagacacttctggattgagagcaatcagaagagagaggagctgggcctggaggggcagctgatactgagaccaagcaatggcatctggatcaagctgaagagaagaaatgctgatgagagataaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcccctaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 16 - DNA, 3961 bp, synthetic construct
  source 1..3961: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctcgaggagcttggcccattgcatacgttgtatccatatcataatatgtacatttatattggctcatgtccaacattaccgccatgttactagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcgcgccgccaccatggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcccctaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - skipped

SEQ ID NO: 18 - DNA, 23 bp, synthetic construct
  source 1..23: mol_type = other DNA
agtatgtccgtgcagtttatagg

SEQ ID NO: 19 - DNA, 23 bp, synthetic construct
  source 1..23: mol_type = other DNA
catacaggtcatcgctgaacggg

SEQ ID NO: 20 - DNA, 23 bp, synthetic construct
  source 1..23: mol_type = other DNA
tcatacaggtcatcgctgaacgg

SEQ ID NO: 21 - DNA, 23 bp, synthetic construct
  source 1..23: mol_type = other DNA
gattatcattcaaatcatacagg

SEQ ID NO: 22 - DNA, 20 bp, synthetic construct
  source 1..20: mol_type = other DNA
gaaatcacactccaccggga

SEQ ID NO: 23 - DNA, 24 bp, synthetic construct
  source 1..24: mol_type = other DNA
acctttactgcttaaacacatgct

SEQ ID NO: 24 - DNA, 20 bp, synthetic construct
  source 1..20: mol_type = other DNA
caggcagcagaaatcgcaag

SEQ ID NO: 25 - DNA, 20 bp, synthetic construct
  source 1..20: mol_type = other DNA
agcctgttcccttcgtcatc

SEQ ID NO: 26 - DNA, 102 bp, synthetic construct
  source 1..102: mol_type = other DNA
taactagggtgcatccaagtccaaacagaagcatgtgattatcattcaaagcgaacgggccaatgaaatgaacgccaatgaagactgtagaggtgatggcag

SEQ ID NO: 27 - protein, 525 aa, synthetic construct
MAGLWLGLVWQKLLLWGAASALSLAGASLVLSLLQRVASYARKWQQMRPIPTVARAYPLVGHALLMKPDGREFFQQIIEYTEEYRHMPLLKLWVGPVPMVALYNAENVEVILTSSKQIDKSSMYKFLEPWLGLGLLTSTGNKWRSRRKMLTPTFHFTILEDFLDIMNEQANILVKKLEKHINQEAFNCFFYITLCALDIICETAMGKNIGAQSNDDSEYVRAVYRMSEMIFRRIKMPWLWLDLWYLMFKEGWEHKKSLQILHTFTNSVIAERANEMNANEDCRGDGRGSAPSKNKRRAFLDLLLSVTDDEGNRLSHEDIREEVDTFMFEGHDTTAAAINWSLYLLGSNPEVQKKVDHELDDVFGKSDRPATVEDLKKLRYLECVIKETLRLFPSVPLFARSVSEDCEVAGYRVLKGTEAVIIPYALHRDPRYFPNPEEFQPERFFPENAQGRHPYAYVPFSAGPRNCIGQKFAVMEEKTILSCILRHFWIESNQKREELGLEGQLILRPSNGIWIKLKRRNADER

SEQ ID NO: 28 - DNA, 1575 bp, synthetic construct
  source 1..1575: mol_type = other DNA
atggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgc

(19) United States
(12) Patent Application Publication
(10) Pub. No.: US 2024/0366794 A1
(43) Pub. Date: Nov. 7, 2024

(54) OPTIMIZED CYP4V2 GENE AND APPLICATION THEREOF

(71) Applicant: SUZHOU NGGT BIOTECHNOLOGY CO.,LTD., Suzhou (CN)

(72) Inventors: Qing CAO, Suzhou (CN); Hai ZHANG, Suzhou (CN); Shengmei WEN, Suzhou (CN); Lixin JIANG, Suzhou (CN)

(21) Appl. No.: 18/751,145

(22) Filed: Jun. 21, 2024

Related U.S. Application Data
(63) Continuation of application No. PCT/CN2022/140586, filed on Dec. 21, 2022.

(30) Foreign Application Priority Data
Dec. 22, 2021 (CN) 202111580873.0

(51) Int. Cl. A61K 48/00; A61K 38/00; A61P 27/02; C12N 9/02; C12N 15/86

(52) U.S. Cl. CPC A61K 48/005; A61P 27/02; C12N 9/0071; C12N 15/86; A61K 38/00; C12N 2740/15043; C12N 2740/16043; C12N 2750/14143; C12N 2770/32044; C12N 2820/60; C12N 2830/003; C12N 2830/48; C12N 2830/50; C12Y 114/14001

(57) ABSTRACT

Provided are an optimized CPY4V2 gene and an application thereof. The gene relates to a polynucleotide for coding a CYP4V2 protein, and comprises a nucleotide sequence having 90% or over 90% of identity with a nucleotide sequence as shown in SEQ ID NO: 5, SEQ ID NO: 6, SEQ ID NO: 7, SEQ ID NO: 8 or SEQ ID NO: 9. Also provided is an expression vector comprising the gene. The expression vector can realize lasting and stable expression in retinal pigment epithelium of eyes, can effectively reduce the dosage and possible side effects of a gene therapy drug for treating BCD, and improves the therapeutic effect.

### TECHNICAL FIELD

The present disclosure is in the field of gene therapy and specifically relates to optimized CYP4V2 gene expression for gene therapy against Bietti crystalline dystrophy (BCD).

### BACKGROUND ART

Bietti crystalline dystrophy (BCD, human Mendelian genetic disease OMIM210370) is a rare hereditary autosomal recessive disease with a biallelic mutation in the CYP4V2 gene. The disease was first described in 1937 by Italian doctor G. B. Bietti. It is characterized by the presence of numerous fine, shiny; yellow-white, crystalline-like deposits at the posterior pole of the retina, atrophy of the retinal pigment epithelium (RPE), pigment clots, and choroidal sclerosis. Progression of the disease ultimately results in decreased vision, night blindness, visual field loss, and impaired color vision. The onset of the disease may occur from adolescence to the age of thirty, but may also occur after the age of thirty: As the disease progresses, loss of peripheral vision, central vision, or both ultimately lead to legal blindness in most patients (Garcia-Garcia, Martinez-Rubio, et al. (2019). “Current perspectives in Bietti crystalline dystrophy.” Clin Ophthalmol 13: 1379-1399. ).

BCD is caused by a mutation in the CYP4V2 gene. This gene is located on the long arm of human chromosome 4 and encodes an important 525 amino acid protein member of cytochrome P450 (family 4, subfamily IV, peptide 2), which is involved in fatty acid metabolism (Li, Jiao et al. (2004). “Bietti crystalline corneoretinal dystrophy is caused by mutations in the novel gene CYP4V2.” Am J Hum Genet 74(5): 817-826). The CYP4V2 protein is present in the epithelial cells of the retina and cornea and the enzyme is localized to the endoplasmic reticulum. This enzyme has the typical omega-hydroxylase activity of CYP4 for medium-chain saturated fatty acids. CYP4V2 is the only CYP4 present at a significant level in retinal cells, and it may be an important contributor to the metabolism of polyunsaturated fatty acids in retinal cells. Gene mutations leading to defective catalytic function of CYP4V2 block degradation of ocular lipids and subsequent accumulation in the eyes of BCD patients (Hata, Ikeda, et al. (2018). “Reduction of lipid accumulation rescues Bietti's crystalline dystrophy phenotypes.” Proc Natl Acad Sci USA 115(15): 3936-3941: Zhang, Yan, et al. (2020). “PSCs Reveal PUFA-Provoked Mitochondrial Stress as a Central Node Potentiating RPE Degeneration in Bietti's Crystalline Dystrophy.” Mol Ther 28(12): 2642-2661). Systemic lipid inclusion was also found in some patients (Lai, Chu, et al. (2010). “Alterations in serum fatty acid concentrations and desaturase activities in Bietti crystalline dystrophy unaffected by CYP4V2 genotypes.” Invest Ophthalmol Vis Sci 51(2): 1092-1097).

It is estimated that one out of every 67,000 people experiences BCD. It is more common in populations of East Asian descent, especially in Chinese and Japanese backgrounds. It is estimated that there are 21,000 patients in China and approximately 5,000 patients in the United States. BCD may be undiagnosed because its symptoms resemble those of other ocular diseases that gradually damage the retina. Currently, there is no treatment available for BCD (Ng, Lai, et al. (2016). “Genetics of Bietti Crystalline Dystrophy.” Asia Pac J Ophthalmol (Phila) 5(4): 245-252: Wang, Chen, et al. (2021). “New compound heterozygous CYP4V2 mutations in bietti crystalline corneoretinal dystrophy: ” Gene 790: 145698.).

BCD's gene therapy strategy is under development. In an HFD-induced BCD mouse model, the human CYP4V2 gene carried by an AAV vector is expressed in the RPE layer by subretinal injection, effectively restoring some physiological and functional defects, conceptually validating gene therapy for BCD disease (Qu, Wu et al. (2020). “Treating Bietti crystalline dystrophy in a high-fat diet-exacerbated murine model using gene therapy.” Gene Ther 27(7-8): 370-382).

Currently; AAV-mediated CYP4V2 gene therapy is at an early stage of research and development in several pharmaceutical companies. To date, there have been no reports proving that CYP4V2 can be fully and continuously expressed in vivo to achieve effective therapeutic effects.

### SUMMARY OF THE INVENTION

To solve the problems existing in the prior art, it is an object of the present disclosure to provide a viral vector for optimizing gene expression to efficiently and persistently, and stably express human CYP4V2 in the retinal RPE layer of the eye and for treating BCD.

In one aspect, the present disclosure provides a polynucleotide encoding a CYP4V2 protein comprising a nucleotide sequence having 90% or more, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 5, SEQ ID NO: 6, SEQ ID NO: 7, SEQ ID NO: 8 or SEQ ID NO: 9.

In some embodiments of the disclosure, the polynucleotide is shown in SEQ ID NO: 5, SEQ ID NO: 6, SEQ ID NO: 7, SEQ ID NO: 8, or SEQ ID NO: 9.

In some embodiments of the disclosure, the polynucleotide is shown in SEQ ID NO: 5.

In another aspect, the present disclosure provides an expression cassette comprising the polynucleotide and a promoter operably linked to the polynucleotide.

In the other hand, the present disclosure provides a vector comprising polynucleotides of the aforementioned aspects, comprising the polynucleotides or the expression cassette.

In some embodiments of the present disclosure, the polynucleotide encoding the CYP4V2 protein is operably connected to an expression control element.

In some embodiments of the present disclosure, the expression control element is selected from one or more of a transcription/translation control signal, an origin of replication, a promoter, an enhancer, an intron, a polyA signal, ITR, an insulator, an RNA processing signal, and an element enhancing the stability of mRNA and/or proteins.

In another aspect, the disclosure provides a cell containing the expression vector.

In another aspect, the disclosure provides a viral particle comprising the expression vector.

In another aspect, the present disclosure provides a pharmaceutical composition for treating BCD comprising the polynucleotide, the expression cassette, the expression vector and/or the viral particle, and a pharmaceutically acceptable carrier, wherein the pharmaceutical composition expresses wild-type or codon-optimized CYP4V2 protein.

In another aspect, the present disclosure provides the use of the polynucleotide, the expression cassette, the expression vector, the viral particle, and/or the pharmaceutical composition for the manufacture of a medicament for the treatment of Bietti crystalline dystrophy (BCD).

In another aspect, the present disclosure provides a method for treating BCD, the method comprising administering to a subject an effective amount of the polynucleotide, the expression cassette, the expression vector, the viral particle, and/or the pharmaceutical composition.

In another aspect, the present disclosure provides a method of constructing a BCD cell model with a CYP4V2 mutation comprising the steps:
(1) designing sgRNA against CYP4V2 gene:
(2) constructing the sgRNA obtained in step (1) into a Cas9-sgRNA vector to obtain a Cas9-sgRNA plasmid: and
(3) introducing the Cas9-sgRNA plasmid obtained in step (2) into a cell to obtain a cell having a CYP4V2 gene mutation.

### BRIEF DESCRIPTION OF THE DRAWINGS

FIG. 1 is a schematic diagram of the structure of a CYP4V2 expression cassette of a recombinant AAV (rAAV) vector disclosed in this disclosure.

FIG. 2 shows the in vitro expression of a codon-optimized CYP4V2 opt gene in plasmid-transfected HEK293 cells. Wherein, (A) a representative image of a CYP4V2 protein Western blot: (B) quantification of CYP4V2 protein Western blot to detect expression levels of CYP4V2 and GFP (plasmid-transfected control) expressed in HEK293 cells with WT or opt genes and GFP expressed using CAG-CYP4V2 WT/opt and CMV-EGFP dual plasmid. Cell lysates were harvested 2 days after transfection of equal amounts of plasmids. Equal amounts of total protein were separated by SDS-PAGE and then immunoblotted. Expression levels of CYP4V2 were normalized with GFP and calculated as a ratio to WT expression level.

FIG. 3 shows the in vitro expression of codon-optimized CYP4V2 in AAV2-transfected ARPE-19 cells. Wherein, (A) a representative image of a CYP4V2 protein Western blot: (B) quantification of CYP4V2 protein Western blot to detect expression levels of CYP4V2 and GAPDH (internal control) in ARPE-19 cells, wherein WT or opt gene or the EGFP control was mediated with equal amounts of AAV2 viral particles (MOI=20000). Cell lysates were harvested 2 days after virus particle infection. Equal amounts of total protein were separated by SDS-PAGE and then immunoblotted. The expression level of CYP4V2 was normalized to GAPDH and calculated as a ratio to the WT expression level (compared to WT expression level: *, p<0.05: **, p<0.01: N=3, student test).

FIGS. 4-6 show the in vitro potency of AAV2-CYP4V2 in the BCD cell model.

FIG. 4 shows the establishment and phenotypic characterization of the HEK293 CYP4V2 mutant cell model. CYP4V2 mutated HEK293 cells exhibit a defective cellular phenotype. Among them, (A) a schematic diagram of the exon 5 mutation of CYP4V2 was generated using CRIPSR/Cas9 method. (B) Sanger sequencing result of CYP4V2 gene targeting site in HEK293 exon 5 mutant cell clone 14 (5-C14). (C) Evaluation of the cell proliferation rate of HEK293 WT and mutant 5-C14 cells during 6 days of culture (compared to WT: **, p<0.01: ***, p<0.005: student test: n=4). (D-E) Evaluation of the autophagy marker LC3B-I/II in HEK293 WT and mutant cells in the presence or absence of treatment with bafilomycin-A1 (Baf, 100 nM, 2 h). (D) Representative images of Western blot. (E) Quantification of LC3B-II protein levels, normalized to GAPDH and calculated as a ratio to WT cells without Baf treatment (NT) (*, p<0.05: ns: no significant difference: student test: n=3).

FIG. 5 shows the establishment of a CYP4V2 mutant ARPE-19 cell model, phenotypic characterization, and the effect of AAV2-CYP4V2 WT and the optimized gene opt18 on the phenotype of the ARPE-19 mutant cell model. The CYP4V2 exon 5 mutation was generated using the CRISPR/Cas9 method. Among them, (A) sanger sequencing of CYP4V2 gene targeting site in ARPE-19 cells. (B) The cell proliferation rate of WT and mutant 5-C13 cells was assessed during a 6-day culture period. ***, P <0.01: student test: n=6. (C) Cellular lipid deposition. 5-C13 cells were infected by WT, mutant 5-C13 cells, AAV2-CYP4V2 WT or opt18 with MOI 20000. Lipids and DAPI staining of cells after arachidonic acid (AA) treatment. Representative image of a laser confocal microscope. Scale bar, 25 microns. (D) Quantification of intracellular fat particle deposition. After infecting mutant cells with AAV2 CYP4V WT and opt18, the deposition of fat particles was significantly reduced. The effect of opt18 on the reduction of fat particle deposition was superior to WT (***, p<0.005: ****, p<0.001: n=50, student test). (E) Electron micrograph of WT and mutant cell 5-C13 showing autophagic vacuoles (black arrows) and vacuoles (white arrows). Scale bar, 500 nm.

FIG. 6 shows the effect of AAV2-CYP4V2 WT and an optimized gene on the phenotype of a HEK293 CYP4V2 mutant cell model. (A) Western blot detection of CYP4V2 protein expression levels in AAV2 CYP4V2 WT or opt18 or EGFP control cells after 2 days of MOI 20000 infection. (B) Evaluation of the cell proliferation rate during a 6-day period after infection. (C) On day 6, cell proliferation in HEK293 mutant cells infected with MOI 5000 or 20000 using AAV2 CYP4V2 opt18 or an EGFP control. (D) On day 6, cell proliferation comparison between wild-type HEK293 cells and HEK293 mutant cells infected with AAV2-CYP4V2 opt18 and EGFP control with MOI 20000 (*, p<0.05: ****, p<0.001: N=6, student test). (E-F) Evaluation of the autophagy marker LC3B-I/II in HEK293 mutant cells infected with AAV2-CYP4V2 WT opt18 and EGFP control with MOI 20000 with or without Baf treatment. (E) Representative images of Western blots. (F) Quantification of LC3B-II protein levels normalized to GAPDH and calculated as a ratio of EGFP NT conditions (*, p<0.05: **, p<0.01: ns: no significant difference: student test: n=3).

FIG. 7 shows in vivo expression of codon-optimized AAV-CYP4V2 in mouse eyes by subretinal injection. (A) AAV2-CYP4V2 wild-type and optimized gene expression in mouse eyes. Wild-type mouse eyeballs were injected through the subretinal space with 1 ul of 5×1012 vg/ml AAV2-CYP4V2 WT or opt recombinant viral particles. After 4 weeks, eye tissues were taken. The retinas were isolated and homogenized for lysis. Equal amounts of total protein were separated by SDS-PAGE and then immunoblotted. (A) Representative image of CYP4V2 protein Western blot: (B) Quantification of CYP4V2 protein Western blot to detect expression levels of CYP4V2 and GAPDH (internal control), the expression level of CYP4V2 being normalized to GAPDH and calculated as a ratio to WT expression level (compared to WT expression level: *, p<0.05: student test: n=6-9). Std: purified recombinant human CYP4V2 protein standard sample. NC: an uninjected mouse eyeball negative control.

### DETAILED DESCRIPTION OF THE INVENTION

In this disclosure, unless otherwise stated, the scientific and technical terms used herein have the meanings commonly understood by those skilled in the art. Also, as used herein, protein and nucleic acid chemistry, molecular biology; cell and tissue culture, microbiology, immunology, and laboratory procedures used herein are terms and routine procedures widely used in the corresponding fields. Meanwhile, in order to better understand the present disclosure, definitions and explanations of related terms are provided below.

As mentioned in this article, “about” a certain value or parameter includes (and describes) implementation solutions for the value or parameter itself. For example, a description referring to “about X” includes a description of “X”.

As used herein, the singular forms “a”, “an”, and “the” include plural referents unless the context clearly dictates otherwise.

A “vector” as used herein refers to a recombinant plasmid or virus comprising a nucleic acid to be delivered into a host cell (in vitro or in vivo).

The term “polynucleotide” or “nucleic acid” as used herein refers to a polymeric form of nucleotides of any length, either a ribonucleotide or a deoxyribonucleotide. Thus, the term includes, but is not limited to, single, double or multi-stranded DNA or RNA, genomic DNA, cDNA, DNA-RNA hybrids or polymers comprising purine and pyrimidine bases or other natural, chemically or biochemically modified, non-natural or derivatized nucleotide bases. The backbone of the nucleic acid may comprise sugar and phosphate groups (as commonly found in RNA or DNA), or modified or substituted sugar or phosphate groups. Alternatively, the backbone of the nucleic acid may comprise a polymer of synthetic subunits such as phosphoramidates and thus may be oligodeoxynucleoside phosphoramidates (P—NH₂) or mixed phosphoramidate-phosphodiester oligomers. In addition, double-stranded nucleic acids can be obtained from single-stranded polynucleotide products that are chemically synthesized (either by synthesizing the complementary strand under appropriate conditions and annealing the strand, or by synthesizing the complementary strand de novo using DNA polymerase with appropriate primers).

A “recombinant viral vector” refers to a recombinant polynucleotide vector comprising one or more heterologous sequences (i.e. nucleic acid sequences of non-viral origin). In the case of recombinant AAV vectors, the recombinant nucleic acid is flanked by at least one, preferably two inverted terminal repeats (ITR).

A “recombinant AAV vector (rAAV vector)” refers to a polynucleotide vector comprising one or more heterologous sequences (i.e. non-AAV derived nucleic acid sequences) flanked by at least one, and preferably two, AAV inverted terminal repeats (ITR). When present in a host cell that has been infected with an appropriate helper virus (or expresses an appropriate helper function) and expresses the AAV rep and cap gene products (i.e. AAV Rep and Cap proteins), the rAAV vector can replicate and package into infected viral particles. When the rAAV vector is incorporated into a larger polynucleotide (e.g. in a chromosome or another vector such as a plasmid for cloning or transfection), the rAAV vector may be referred to as a “pro-vector” which can be “rescued” by replication and encapsidation in the presence of the AAV packaging function and appropriate helper functions. The rAAV vector can be any of a variety of forms, including but not limited to a plasmid, a linear artificial chromosome, which can be complexed with a liposome, encapsulated within a liposome, and in embodiments, encapsulated in a viral particle, particularly an AAV particle. rAAV vectors can be packaged into an AAV viral capsid to produce “recombinant adeno-associated virus particles (rAAV particles)”. AAV helper functions (i.e. functions that allow AAV replication and packaging from a host cell) can be provided in any of a variety of forms, including, but not limited to, helper viruses or helper viral genes that facilitate AAV replication and packaging. Other AAV helper functions are known in the art.

“rAAV virus” or “rAAV viral particle” refers to a viral particle consisting of at least one AAV capsid protein and an encapsidated rAAV vector genome.

“heterologous” means an entity that differs in genotype from the rest of the entity to which it is compared or into which it is introduced or incorporated. For example, nucleic acids introduced into different cell types by genetic engineering techniques are heterologous nucleic acids (and, when expressed, may encode heterologous polypeptides). Similarly, a cellular sequence (e.g. a gene or portion thereof) incorporated into a viral vector is a heterologous nucleotide sequence with respect to the vector.

As used in reference to viral titer, the term “genomic particle (gp)”, “genomic equivalent” or “genomic copy” refers to the number of virions comprising the recombinant AAV DNA genome, whether infectious or functional. The number of genomic particles in a particular vector preparation can be determined by methods such as those described in the examples herein or, for example, by Clark et al. (1999) Hum. Gene Ther. 10: 1031-1039: Veldwijk et al. (2002) Mol. Ther. 6:272-278.

As used in reference to viral titer, the term “infectious unit (iu)”, “infectious particle” or “replication unit” refers to the number of recombinant AAV vector particles that have the ability to infect and replicate as measured by an infection center assay, also referred to as a replication center assay; as described, for example, in Mclaughlin et al. (1988) J. Virol. 62: 1963-1973.

As used in reference to viral titer, the term “transduction unit (tu)” refers to the number of infectious recombinant AAV vector particles that result in the production of a functional transgene product, as measured in a functional assay, as exemplified herein or, for example, in Xiao et al. (1997) Exp. Neurobiol., 144:113-124; or Fisher et al. (1996) J. Virol., 70:520-532 (LFU assay).

An “inverted terminal repeat” or “ITR” sequence is a term well-known in the art and refers to a relatively short sequence found at the end of the viral genome, in the opposite direction.

An “AAV inverted terminal repeat (ITR)” sequence is a well-known term in the art and is a sequence of about 145 nucleotides found at both ends of the native single-stranded AAV genome. The outermost 125 nucleotides of an ITR can exist in either of two alternative orientations, resulting in heterogeneity between different AAV genomes and between the two ends of a single AAV genomc. The outermost 125 nucleotides also contain several shorter regions that are self-complementary (referred to as the A, A′, B, B′, C, C′ and D regions), allowing interstrand base pairing to occur within the ITR portion.

A “helper virus” for AAV refers to a virus that allows AAV (which is a defective parvovirus) to replicate and package through a host cell. A variety of such helper viruses have been identified, including adenoviruses, herpes viruses, and poxviruses such as vaccinia. Adenoviruses encompass a number of different subtypes, although adenovirus type 5 (Ad5) of subtype C is most commonly used. A variety of adenoviruses of human, non-human mammalian, and avian origin are known and available from depositories such as ATCC. The herpes virus family, which is also available from depositories such as ATCC, includes for example herpes simplex virus (HSV), Epstein-Barr viruses (EBV), cytomegaloviruses (CMV), and pseudorabies viruses (PRV).

The “percentage (%) sequence identity” relative to the reference peptide or nucleic acid sequence is defined as the percentage of amino acid residues or nucleotides in the candidate sequence that are identical to the amino acid residues or nucleotides in the reference peptide or nucleic acid sequence after aligning the sequence and introducing gaps (if necessary; to achieve maximum percentage sequence identity without considering any conservative substitutions as part of sequence identity). Alignment for purposes of determining percent amino acid or nucleic acid sequence identity can be accomplished in a variety of ways in the art, for example, using publicly available computer software programs, such as those described in Current Protocols in Molecular Biology (Ausubel et al. cds. 1987), Supp. 30, section 7.7.18, Table 7.7.1, and including BLAST, BLAST-2, ALIGN, or the Megalign (DNASTAR) software. A preferred alignment software is ALIGN Plus (Scientific and Educational Software, Pennsylvania). Those skilled in the art can determine appropriate parameters for measuring alignment, including any algorithm that needs to achieve maximum alignment over the full length of the compared sequence. For purposes herein, the % amino acid sequence identity of a given amino acid sequence A to, with, or against a given amino acid sequence B (which may alternatively be referred to as amino acid sequence A having or comprising a certain % amino acid sequence identity to, with or against a given amino acid sequence B) is calculated as follows: 100 times the score X/Y, where X is the number of amino acid residues in the program alignment of A and B that are scored as identical matches by the sequence alignment program, and where Y is the total number of amino acid residues in B. It will be appreciated that where the length of amino acid sequence A is not equal to the length of amino acid sequence B, the % amino acid sequence identity of A to B will not be equal to the % amino acid sequence identity of B to A. For purposes herein, the % nucleic acid sequence identity of a given nucleic acid sequence C to, with, or against a given nucleic acid sequence D (which may alternatively be referred to as a given nucleic acid sequence C having a certain % nucleic acid sequence identity to, with or against a given nucleic acid sequence D) is calculated as follows: 100 times the score W/Z, where W is the number of nucleotides scored as identical matches by the sequence alignment program in the programmed alignment of C and D, and where Z is the total number of nucleotides in D. It will be appreciated that where the length of nucleic acid sequence C is not equal to the length of nucleic acid sequence D, the % nucleic acid sequence identity of C to D will not be equal to the % nucleic acid sequence identity of D to C.

The “effective amount” is the amount that is sufficient to affect beneficial or expected outcomes, including clinical outcomes (such as improving symptoms, achieving clinical endpoints, etc.). An effective amount can be administered once or multiple times. For disease states, an effective amount is an amount sufficient to ameliorate, stabilize, or delay disease progression. For example, an effective amount of rAAV particles expresses a desired amount of a heterologous nucleic acid such as a therapeutic polypeptide or therapeutic nucleic acid.

The “individual” or “subject” is a mammal. Mammals include but are not limited to, domesticated animals (e.g. cows, sheep, cats, dogs, and horses), primates (e.g. humans and non-human primates such as monkeys), rabbits, and rodents (e.g. mice and rats). In some embodiments, the individual or subject is a human.

“Treatment” as used herein is a means for obtaining beneficial or desired clinical results. For purposes of this disclosure, beneficial or expected clinical results include but are not limited to, amelioration of symptoms, diminishment of extent of disease, stabilization (such as non-worsening) of the disease, prevention of disease spread (such as metastasis), delay or alleviation of disease progression, improvement or alleviation of disease status, and remission (whether partial or total), whether detectable or undetectable. “Treatment” may also mean prolonging survival as compared to expected survival if not receiving treatment.

In one aspect, the present disclosure provides a polynucleotide encoding a CYP4V2 protein comprising a nucleotide sequence having 90% or more, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 5, SEQ ID NO: 6, SEQ ID NO: 7, SEQ ID NO: 8 or SEQ ID NO: 9.

In some embodiments of the disclosure, the polynucleotide is as shown in SEQ ID NO: 5, SEQ ID NO: 6, SEQ ID NO: 7, SEQ ID NO: 8, or SEQ ID NO: 9.

In another aspect, the present disclosure provides an expression cassette comprising the polynucleotide and a promoter operably linked to the polynucleotide.

In another aspect, the present disclosure provides an expression vector comprising the polynucleotide or the expression cassette.

In some embodiments of the present disclosure, the polynucleotide encoding the CYP4V2 protein is operably connected to an expression control element.

In some embodiments of the present disclosure, the expression control element is selected from one or more of a transcription/translation control signal, an origin of replication, a promoter, an enhancer, an intron, a polyA signal, ITR, an insulator, an RNA processing signal, and an element enhancing the stability of mRNA and/or proteins.

In some embodiments of the present disclosure, the expression vector comprises an origin of replication: preferably; the origin of replication sequence is selected from the group consisting of f1 bacteriophage ori, RK2 oriV, pUC ori, and pSC101 ori.

In some embodiments of the present disclosure, the expression vector further comprises a 5′ ITR: preferably, the 5′ITR comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 1: more preferably, the polynucleotide is as shown in SEQ ID NO: 1.

In some embodiments of the present disclosure, the expression vector further comprises a 3′ ITR: preferably, the 3′ ITR comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 11: more preferably, the polynucleotide is as shown in SEQ ID NO: 11.

In some embodiments of the present disclosure, the expression vector further comprises an enhancer: preferably, the enhancer is a CMV enhancer: more preferably, the enhancer comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 2: more preferably; the polynucleotide is as shown in SEQ ID NO: 2.

In some embodiments of the present disclosure, the expression vector further comprises a promoter. In some embodiments of the present disclosure, the promoter is a specific or non-specific promoter. In some embodiments of the present disclosure, the promoter is selected from a CBA promoter, CMV promoter, SV40 promoter, hPGK promoter, and TRE 3GS promoter. In some embodiments of the present disclosure, the promoter is a CBA promoter: preferably the CBA promoter comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 3. In some embodiments of the disclosure, the polynucleotide is as shown in SEQ ID NO: 3. In some embodiments of the present disclosure, the promoter is an inducible promoter. Preferably, the inducible system comprises one or more of a tetracycline-regulated promoter, an alcohol-regulated promoter, a steroid-regulated promoter, a metal-regulated promoter, a pathogenicity-regulated promoter, a temperature/heat-inducible promoter, and a light-regulated promoter, an and IPTG inducible system. In some embodiments of the disclosure, the tetracycline-regulated promoter is selected from a Tet on promoter, a Tet off promoter, and a Tet Activator promoter. In some embodiments of the present disclosure, the alcohol-regulated promoter is selected from an alcohol dehydrogenase I (alcA) gene promoter, a promoter responsive to an alcohol transactivator protein (AlcR). In some embodiments of the present disclosure, the steroid-regulated promoter is selected from a rat glucocorticoid receptor promoter, a human estrogen receptor promoter, a moth ecdysone receptor promoter, a retinoid promoter, and a thyroid receptor superfamily promoter. In some embodiments of the present disclosure, the metal-regulated promoter is selected from yeast, mouse, and human metallothionein promoters. In some embodiments of the present disclosure, the pathogenic-regulated promoter is selected from a salicylic acid-regulated promoter, an ethylene-regulated promoter, and a benzothiadiazole-regulated (BTH) promoter. In some embodiments of the present disclosure, the temperature/heat-inducible promoter is selected from an HSP-70 promoter, an HSP-90 promoter, and a soybean heat shock promoter. In some embodiments of the present disclosure, the light regulatory promoter is a light-responsive promoter of plant cells.

In some embodiments of the present disclosure, the expression vector further comprises an exon and an intron. In some embodiments of the present disclosure, the exon and intron are the first exon and first intron of the chicken beta-actin gene. In some embodiments of the present disclosure, the exon and intron include a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 4. In some embodiments of the disclosure, the polynucleotide is as shown in SEQ ID NO: 4.

In some embodiments of the present disclosure, the expression vector further comprises a polyA signal. In some embodiments of the present disclosure, the polyA signal is bovine growth hormone poly A (BGH poly A), short poly A, SV40 polyA, and/or human beta-globin poly A. In some embodiments of the present disclosure, the polyA signal comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 10. In some embodiments of the disclosure, the polynucleotide is as shown in SEQ ID NO: 10.

In some embodiments of the present disclosure, the expression vector comprises a nucleotide sequence having 90% or higher identity, preferably a nucleotide sequence having 91%, 92%, 93%, 94%, 95%, 96%, 97%, 98%, 99% or higher identity, more preferably a nucleotide sequence having 98%, 99%, or higher identity to the nucleotide sequence as shown in SEQ ID NO: 12, SEQ ID NO: 13, SEQ ID NO: 14, SEQ ID NO: 15, and SEQ ID NO: 16. In some embodiments of the disclosure, the polynucleotide is as shown in SEQ ID NO: 12, SEQ ID NO: 13, SEQ ID NO: 14, SEQ ID NO: 15 or SEQ ID NO: 16.

In some embodiments of the present disclosure, the vector comprises a post-transcriptional regulatory element. In some embodiments of the present disclosure, the post-transcriptional regulatory element is a woodchuck hepatitis virus post-transcriptional regulatory element (WPRE).

In some embodiments of the present disclosure, the vector further comprises a gene encoding a marker. In some embodiments of the present disclosure, the marker is selected from one or more of an antibiotic resistance protein, a toxin resistance protein, a colored or fluorescent or luminescent protein, and a protein mediating enhanced cell growth and/or gene amplification.

In some embodiments of the present disclosure, the antibiotic is selected from ampicillin, neomycin, G418, puromycin, and blasticidin.

In some embodiments of the present disclosure, the toxin is selected from anthrax toxin and diphtheria toxin.

In some embodiments of the present disclosure, the colored or fluorescent, or luminescent protein is selected from a green fluorescent protein, an enhanced green fluorescent protein, a red fluorescent protein, and luciferase. In some embodiments of the present disclosure, the protein mediating enhanced cell growth and/or gene amplification is dihydrofolate reductase (DHFR).

In some embodiments of the present disclosure, the expression vector is selected from a plasmid, cosmid, viral vector, RNA vector, or linear or circular DNA or RNA molecule.

In some embodiments disclosed herein, the plasmid is selected from pCI, puc57, pcDNA3, pSG5, pJ603, and pCMV.

In some embodiments of the present disclosure, the viral vector is selected from retrovirus, adenovirus, parvovirus (e.g. adeno-associated virus), coronavirus, negative-stranded RNA virus such as orthomyxovirus (e.g. influenza virus), rhabdovirus (e.g. rabies and vesicular stomatitis virus), paramyxovirus (e.g. measles and Sendai virus), positive-stranded RNA virus such as RNA parvoviruses and alphaviruses, and double-stranded DNA virus including adenoviruses, herpes virus (e.g. herpes simplex virus types 1 and 2, Epstein-Bar virus, cytomegalovirus) and poxviruses (e.g. cowpox virus, fowlpox virus, and canarypox virus), norwalk virus, togavirus, flavivirus, reovirus, papovavirus, hepadnavirus, baculovirus, and hepatitis virus.

In some embodiments of the present disclosure, the retrovirus is selected from avian leukosis-sarcoma, mammalian C-type, B-type viruses, D-type viruses, HTLV-BLV aggregate, lentivirus, and spumavirus.

In some embodiments of the present disclosure, the lentiviral vector is selected from HIV-1, HIV-2, SIV, FIV, BIV, EIAV, CAEV, and ovine demyelinating leukoencephalitis lentivirus.

In some embodiments of the present disclosure, the expression vector is an adeno-associated virus.

In some embodiments of the present disclosure, the adeno-associated virus is selected from AAV type 1, AAV type 2, AAV type 3, AAV type 4, AAV type 5, AAV type 6, AAV type 7, AAV type 8, AAV type 9, AAV type 10, avian AAV, bovine AAV, canine AAV, equine AAV, ovine AAV, and the like.

In some embodiments of the disclosure, the expression vector further comprises a shortened chimeric intron and a Kozak initiation sequence.

In some embodiments of the disclosure, the shortened chimeric intron is as shown in SEQ ID NO: 4.

In some embodiments of the present disclosure, the Kozak initiation sequence is as shown in SEQ ID NO: 17.

On the other hand, the present disclosure provides a viral particle comprising the expression vector.

In another aspect, the present disclosure provides a pharmaceutical composition for treating BCD comprising the polynucleotide, the expression cassette, any of the expression vectors and/or the viral particle, and a pharmaceutically acceptable carrier, wherein the pharmaceutical composition expresses wild-type or codon-optimized CYP4V2 protein.

In another aspect, the present disclosure provides the use of the polynucleotide, the expression cassette, the expression vector, the viral particle, and/or the pharmaceutical composition for the manufacture of a medicament for the treatment of Bietti crystalline dystrophy (BCD).

In another aspect, the present disclosure provides a method for treating BCD, the method comprising administering to a subject an effective amount of the polynucleotide, the expression cassette, the expression vector, the viral particle, and/or the pharmaceutical composition.

In another aspect, the present disclosure provides a method of constructing a BCD cell model with a CYP4V2 mutation comprising the steps:
(1) designing sgRNA against CYP4V2 gene:
(2) constructing the sgRNA obtained in step (1) into a Cas9-sgRNA vector to obtain a Cas9-sgRNA plasmid:
(3) introducing the Cas9-sgRNA plasmid obtained in step (3) into a cell to obtain a cell having a CYP4V2 gene mutation.

In some embodiments of the present disclosure, the method further comprises the step of (4) screening for cells having the CYP4V2 gene mutation.

In some embodiments of the present disclosure, the method further comprises the step of (5) identifying the CYP4V2 gene mutant cell.

In some embodiments of the present disclosure, the identification is selected from a cell proliferation assay, an autophagy change assay, and a fat particle deposition assay.

In some embodiments of the present disclosure, the sgRNA is selected from one or more of SEQ ID NOs: 18-21.

The present disclosure provides an adeno-associated viral vector expressing the codon-optimized CYP4V2 gene for efficient, sustained, and stable expression in the retinal pigment epithelial layer of the eye. The present disclosure can effectively reduce the dosage and possible side effects of gene therapy drugs for treating BCD, and improve the therapeutic effect.

For purposes of clarity and conciseness, features are described herein as part of the same or separate embodiments, however, it will be understood that the scope of the disclosure may include some embodiments having a combination of all or some of the features described.

The present disclosure is described in more detail below with reference to specific examples, which, however, are for illustrative purposes only and are not intended to limit the present disclosure.

### EXPERIMENTAL MATERIALS AND METHODS

### CYP4V2 Expression Cassette and AAV Vector

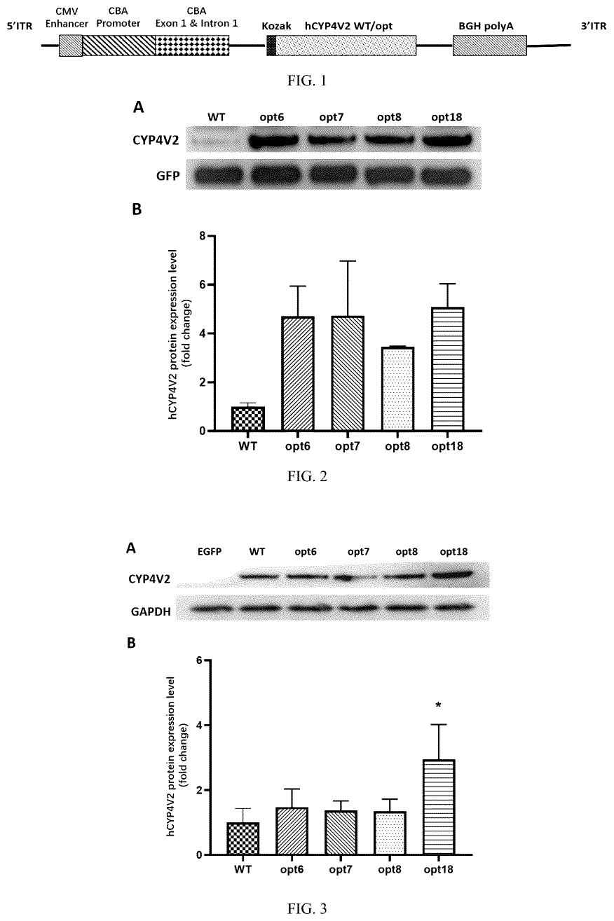

All DNA sequences used in this study were synthesized by the Genscript. Codon optimization was improved using the GenSmart codon optimization tool. The AAV vector-mediated CYP4V2 expression cassette is shown in FIG. 1. The expression cassette sequence was cloned into a shuttle plasmid vector to obtain a shuttle plasmid containing the target gene CYP4V2 mediated by the AAV vector.

### AAV Vector Production and Purification Method

The production of AAV vector uses a three-plasmid system, i.e. a shuttle plasmid containing the target gene CYP4V2, a pRepCap plasmid with the repcap gene of the AAV vector, and a helper plasmid pHelper, and uses PEI as a transfection reagent to co-transfect HEK293 cells and recombine to package an AAV virus vector. Harvesting was performed 48-72 hours after transfection, and the harvest solution was purified to obtain a recombinant AAV virus vector with a certain purity: The purification method was as follows:

The harvest fluid was first pretreated. The HEK293 cells were sufficiently lysed to release the intracellular AAV viral vector. The nuclease was added at the same time to digest the free nucleic acid. After digestion, the macromolecular impurities and cell debris were removed by deep filtration. The filtrate after deep filtration was subjected to secondary filtration to obtain a clear solution for affinity loading.

Affinity chromatography used the specific adsorption of a ligand and a protein to capture the AAV virus vector in the harvest solution, and removes most process-related impurities, achieving the effect of concentration and impurity removal. The collected eluate was mixed well and neutralized with a neutralization buffer and stored in a sterile stock solution bottle as an anion chromatography loading solution.

Anion chromatography used the isoelectric point difference of different components to separate solid and empty-shelled AAV viruses while continuing to remove the residual impurities. The eluent was collected in a new sterile liquid storage bottle. The buffer solution was replaced by the ultrafiltration concentration method to be the buffer solution with stable preparation, while the virus titer was concentrated to about 5×10¹² vg/mL. Finally, the solution was sterilized, filtered, and dispensed for future use.

### Titer Quantification of AAV Vector Stocks

After AAV virus purification was complete, the amount of virus needed to be determined. The genomic titer is the most classical standard for characterizing the physical titer of AAV. Designing primer probes against the genomic sequence of rAAV followed by Q-PCR detection is the most common method for genomic titer detection.

In view of the codon optimization of the ORF coding frame in the present disclosure, involving screening of multiple vector structures, the consensus sequences in the vectors are selected to design primer probes in order to ensure quantitative stability and accuracy between different vector structures. The CMV enhancer sequence is a portion of the CAG promoter that is common to different vector constructs during codon optimization, so primer probes are designed for this sequence.

In the process of genome titer detection, the standard curve shall be established first. The positive standard plasmid shall be diluted to 2×10⁷, 2×10⁶, 2×10⁵, 2×10⁴, 2×10³, and 2×10² copies/μl using a sample diluent as a template of the standard curve. The linearity and amplification efficiency of the standard curve shall be controlled. Generally, R2>0.99, and the amplification efficiency shall be between 90%-110%. Then, the pretreated rAAV sample was diluted and then subjected to QPCR detection, so as to ensure that the Ct value detected by the sample is within the range of the standard curve. The genome titer of the rAAV sample was calculated by substituting the Ct value of the sample into the standard curve, and the content of the product was identified.

### In Vitro Cell Plasmid Transfection Experiment

HEK293 cells were digested one day in advance and seeded into a 6-well plate at 7.0*10⁵ cells/well. The plasmid transfection experiment was performed after overnight incubation. The plasmid was mixed with the transfection reagent in the following amounts: To a 1.5 ml centrifuge tube, 125 μl of opti-MEM medium (Gibco, 31985-070), 2 μg of CYP4V2-opt/WT expression plasmid, 200 ng of CMV-EGFP plasmid and 5 μl of P3000 were add and mixed well to obtain a plasmid tube. To another 1.5 ml centrifuge tube, 125 μl of opti-MEM medium and 5 μl of Lipo 3000 (Thermo, L3000015) were added, mixed well, and then added into the plasmid tube. Standing incubation was performed for 15 min at room temperature. The mixed solution was slowly dropped into HEK293 cells of the 6-well plate, and continued to culture in a CO₂ constant temperature incubator for 48 h.

### In Vitro Cell Virus Infection Experiment

ARPE-19 cells were digested one day in advance and seeded into a 6-well plate at 3.0*10⁵ cells/well. The AAV2 virus infection experiment was performed after overnight incubation for 24 h. One well of cells was counted after digestion. The number of required virus Vg was calculated according to the 20000 MOI infection parameter, and the required amount of virus was diluted to 1 ml with Opti-MEM medium. Then, the cell culture medium in the 6-well plate inoculated overnight was cleaned and 1 ml of diluted virus solution was added. The culture was continued in the CO₂ constant temperature incubator. After 4 h, the system was supplemented with 1 ml of DMEM complete medium (DMEM+10% FBS+1% dual antibody medium (Hyclone, SV30010)), and placed in the CO₂ constant temperature incubator for culture for 48 h.

### Construction of BCD Cell Model with CYP4V2 Mutation

The CRISPR-Cas9 method was used to construct the BCD cell model with CYP4V2 mutation. The specific method was as follows: HEK293 or ARPE-19 cells were subjected to digestion treatment one day in advance and inoculated into a 6-well plate at 7.0*10⁵ cells/well. After 24 h, the plasmid transfection experiment was performed. The plasmid sample dilution was prepared: into a 1.5 ml centrifuge tube, 125 μl of opti-MEM medium, 2 μg of Cas9-sgRNA plasmid (addgene, #58766), and 5 μl of P3000 (Exon7-sgRNA1-HDR repair construction supplemented with 3 μg of CYP4V2-ssDNA) were added and mixed well. Lipo 3000 (Thermo, L3000015) dilution was prepared: into a 1.5 ml centrifuge tube, 125 μl of opti-MEM medium and 5 μl of Lipo 3000were added, mixed well, and added into the plasmid sample dilution tube. After mixing well, incubation was performed for 15 min at room temperature. The mixed solution was slowly dropped into the cells to be transfected of the 6-well plate, gently shaken for mixing well, and placed in a CO₂ constant temperature incubator for culture. 48 H after transfection, fluorescence photography was taken to confirm the transfection efficiency. The cells in the 6-well plate were digested, and DMEM complete medium containing 5 μg/ml Puromycin was added for pressure culture, during which the medium was changed every day, and the medium was DMEM-Full medium containing 5 μg/ml puromycin. After 72 h of culture, the cell culture medium in the 6-well plate was discarded, and the DMEM medium without puromycin was added for culture. After 48 h of culture, the cells were digested and counted, and sorted 1 cell/well into a 96-well plate (containing 200 μl of DMEM+20% FBS+1% dual-antibody medium). After 2-3 weeks of culture, clonal plaques were observed to grow up and step-wisely expanded based on 96 well plate→24 well plate→6 well plate. After the cells in the 6-well plate were completely grown, partial cells were taken to extract the cell genome DNA (Tiangen, DP304-03), which was amplified by PCR. After gel cutting and recycling. Sanger sequencing was performed for validation. The genotype of monoclonal cells was analyzed using SeqMan software. The remaining cells were expanded and cryopreserved according to the needs of the experiment.

### Western Blot Analysis

200 μl of RIPA lysis solution containing a protease inhibitor (Beyotime Biotechnology Co. Ltd. P0013 B) was added to each well of a 6-well plate seeded with cultured cells, and the cells were lysed thoroughly on ice for 5 min. The cell lysate was collected in a 1.5 ml centrifuge tube, and placed for 30 min on ice. After 20 min of centrifuge at 15000 g at 4° C., the supernatant was taken. The protein sample was diluted 10-fold with PBS and mixed well. Protein quantification was performed using the BCA protein quantification kit (Pierce™ BCA protein detection kit (Thermo Fisher, 23225). 5×SDS loading buffer was added to the protein sample and mixed well. 5 min of boiling was performed at 100° C. 30 μg protein sample was separated by SDS-PAGE electrophoresis and transferred to a PVDF membrane. After blocking with 5% skim milk (PBS) for 1 h at room temperature, the antibody was incubated and detected by ECL. Imaging analysis was performed by the Bio-Rad ChemiDoc™ Touch Imaging System.

### Cell Proliferation Analysis

CYP4V2 mutated cells were digested with wild-type cells and counted. The cell suspension with adjusted cell density was mixed well and inoculated into a 96-well plate, with four duplicate wells for each cell. In in vitro efficacy experiments, virus infection was performed 24 h after seeding plates. The CCK-8 assay was performed 48 h after seeding and the days of the assay were day 2, day 3, day 4, day 5, and day 6. On the day of the assay, the number of total wells to be analyzed was counted and an appropriate amount of DMEM medium containing CCK-8 reagent was prepared. The amount of CCK-8 reagent (Dojindo Chemical Co. CK04) and DMEM medium was prepared in a ratio of 1:10. 100 μl of DMEM medium containing CCK-8 reagent was added to each well. After 1.5 h of incubation at 37° C. in a cell culture incubator, the plate was read at 450 nm using a multi-purpose microplate reader (BioTek, SYNERGY/LX). Statistical analysis of the data was performed using GraphPad Prism software.

### Autophagosome Function Detection

Wild-type and mutant cells and virus-infected cells were treated with a culture medium containing 100 nM Bafilomycin-A1 (Baf) or without the drug for 2 h, respectively. Cell proteins were extracted, and the ratio of LC3B-II/GAPDH was analyzed by WB assay to determine whether intracellular autophagy was unobstructed.

### Lipid Deposition Staining

Cells were treated with 160 μM arachidonic acid (AA) for 4 consecutive days and fixed with 4% paraformaldehyde at room temperature for 20 min. Cells were stained with 3 μM BODIPY 493/503 at 37° C. for 20 min. 10 Min of counterstaining was performed with 5 μg/ml DAPI. Photographs were taken with a laser confocal microscope. For each experimental group, 50 cells were selected, and the area of lipid deposition particles inside cells and the number of lipid deposition particles per unit area inside cells were analyzed using the Image pro plus software.

### Subretinal Injection in Mice

Mydriasis was fully dilated after general anesthesia in mice. The sclera was punctured with a 30 ½gauge disposable sharp needle outside the limbus of the corneosclera under direct vision of a special ophthalmological operating microscope to avoid injury to the iris and lens. Then a micro loader with a 33-gauge flat needle was introduced along the puncture site. The needle bypassed the lens and reached the vitreous body, and then gradually inserted into the potential retinal compartment between the neuroretinal layer and the retinal pigment epithelium (RPE) layer and slowly injected, wherein the injection volume was 1 μl of 0.1% Fluorescein sodium dye (safe concentration) was added into the injection vehicle suspension, so as to make it convenient to observe the success of the injection and the scope of retinal detachment. During the injection, 2.5% hydroxypropyl methylcellulose was dropped on the ocular surface to facilitate observation of the fundus at any time. The injection was successful as evidenced by a clear round bulge of the retina in the fundus and green color beneath the bulge under the operating microscope. After a certain time, the bleb disappeared, and the local retinal bulge became flat. Intraoperatively, if the clear round bulge of the retina in the fundus and green color beneath the bulge cannot be seen or if complications such as massive retinal hemorrhage can be seen, the mice will be re-injected. 1% Atropine eye ointment and tetracycline cortisone eye ointment were applied at the end of surgery and repeated every other day for three times to reduce inflammatory reaction and prevent infection.

### Extracting Mouse Eyeball Retina Sample

Mouse eye tissues were taken, cornea and lens were removed. 100 μl of ATL solution (QIAGEN, 19076) was added to homogenize for 2 min. DNA Sample Extraction: 10 μl of the homogenate was aspirated: 10 μl of ATL solution and 2 μl of proteinase K (QIAGEN, 19133) were added to mix well: 20 μl of AL solution (QIAGEN, 19075) was added to mix well: incubation was performed at 56° C. for 10 min to prepare the DNA detection sample. Protein extraction: the remaining 50 μl of homogenate was pipetted: 5 μl of 10 * RIPA lysis solution (CST) and protease inhibitor (Beyotime) were added and mixed well; the lysis was performed for 30 min on ice: after 30 min of centrifugation at 15000 g at 4° C., the supernatant was collected for protein quantification and WB detection.

### Vector Genome Copy Number Analysis

5 μl of DNA detection sample was taken. 45 μl of sample diluent (5 μl of Pluronic F68 (Gibco, 24040032) and 2 μl of tRNA (Ambion, AM7119) were added in 1 ml of RNA enzyme-free water. After mixing well, 5 μl of the diluent was aspirated, added with 45 μl of the sample diluent, and mixed well to obtain a 100-fold diluted DNA sample to be tested. Plotting of standard curves was performed with a linearized and absolutely quantified plasmid (pAAV-CMV-EGFP) as a standard. The qPCR reaction solution was prepared according to the following reaction system: Taqman PCR Mix, upstream primer (10 82 M), downstream primer (10 μM), probe (10 μM), DNA template. The qPCR reaction was carried out under the following reaction conditions: 50° C., 2 min: 95° C., 10 min: 95° C., 15s, 60° C., 30 s, 40 cycles: 37° C., 2 s. At the end of the reaction, a standard curve was prepared according to the Ct value of the standard, and the Vg number of each sample to be tested was calculated by linear regression.

### Mouse OCT Assay

Optical coherence tomography (OCT) examination: the anesthetized animal was placed on a lifting platform, subjected to conventional mydriasis by using compound tropicamide eye drops, and subjected to topical anesthesia by using proparacaine hydrochloride eye drops. The eye position was placed. After mydriasis, medical carbomer eye drops gel was applied to the cornea of the eye to be tested. The light source of the ophthalmic ultramicroscopic imaging system was adjusted and focused. The lens was adjusted to focus on the retina to perform optical coherence tomography (OCT) on the retina. Photographs were taken of both eyes of each experimental animal. After completion of the experiment, both eyes were washed with normal saline, and levofloxacin eye drops were used to prevent infection.

### Mouse ERG Assay

The mice to be tested were placed in the dark room and reared for 3 days to reach the experimental time point. The mice were subjected to waveform acquisition of electroretinogram (ERG) in the dark room. After the mice were anesthetized and mydriasis, 0.25% hydroxypropyl methylcellulose solution was injected into both eyes to protect the cornea. The mice were placed on the operating table with a heating pad. A red metal wire electrode was inserted under the skin epidermis at the center between the two eyes of the mice, and a black ground electrode was inserted under the tail epidermis of the mice. The ERG images were collected by an electroretinogram Ganzfeld ERG system (Micron IV, Phoenix Research Laboratories, Inc), which is automatically analyzed by the software in the system to obtain the average amplitude of a-wave and b-wave for each stimulation intensity for statistical analysis. The waveform of electroretinogram can reflect the conduction function of retinal neurons, and the measured waveform mainly consists of negative a-wave and positive b-wave, wherein the a-wave reflects the function of primary retinal neurons, namely photoreceptor cells, and the b-wave reflects the function of secondary retinal neurons, namely bipolar cells.

### EXAMPLES

### Example 1: Construction and Isolation and Purification of Adenoviral Vector

### 1.1 Construction of Adenoviral Vector

The structure of the CYP4V2 expression cassette is shown in FIG. 1. The CYP4V2 expression cassette comprises, from the 5′ to the 3′ end, a 5′ ITR, a CMV enhancer, a CBA promoter, a CBA exon 1 & intron 1, a Kozak sequence, and a gene of interest: wild-type human CYP4V2 gene hCYP4V2 WT or optimized CYP4V2 gene hCYP4V2 opt, BGH polyA) and 3′ ITR. wherein:

The nucleotide sequence of 5′ ITR is shown as SEQ ID NO: 1;

The CMV enhancer is a cytomegalovirus (CMV) enhancer element, the nucleotide sequence of which is shown in SEQ ID NO: 2;

The CBA promoter is a chicken β-actin gene promoter, the nucleotide sequence of which is shown in SEQ ID NO: 3;

CBA exon 1 & intron 1 is the first exon and the first intron of the chicken β-actin gene, the nucleotide sequence of which is shown in SEQ ID NO: 4;

The hCYP4V2WT gene was derived from the wild-type human CYP4V2 gene (Gene ID: 285440), the gene sequence of which is shown in SEQ ID NO: 9, and the wild-type hCYP4V2 WT protein encoded by it has an NCBI accession number of NP_997235.3.

Optimized CYP4V2 gene hCYP4V2 opt are codon-optimized genes opt18, opt7, opt8, and opt6 encoding the wild-type hCYP4V2 WT protein, and the nucleotide sequences are SEQ ID NO: 5, SEQ ID NO: 6, SEQ ID NO: 7 and SEQ ID NO: 8 respectively.

BGH polyA is a bovine growth hormone polyadenylation signal, the nucleotide sequence of which is shown as SEQ ID NO: 10.

The nucleotide sequence of 3′ ITR is shown as SEQ ID NO. 11.

The Kozak sequence is inserted in front of the CYP4V2 cDNA sequence, for example, SEQ ID NO: 17 (GCCACC).

The expression cassette comprising the wild-type human CYP4V2 gene hCYP4V2WT was constructed as plasmid pAAV2-CYP4V2 WT.

The expression cassette containing the optimized CYP4V2 gene was constructed as plasmid pAAV2-CYP4V2 opt. Among them, SEQ ID NO: 12 shows the nucleotide sequence of the expression cassette comprising the codon-optimized gene opt6: SEQ ID NO: 13 shows the nucleotide sequence of the expression cassette comprising the codon-optimized gene opt7; and SEQ ID NO: 14 shows the nucleotide sequence of the expression cassette comprising the codon-optimized gene opt8: the nucleotide sequence of the expression cassette comprising the codon-optimized gene opt18 is shown in SEQ ID NO: 15. The nucleotide sequence of the expression cassette comprising CYP4V2 WT is shown in SEQ ID NO: 16.

The CYP4V2 expression cassette may be packaged in rAAV with a vector having a capsid from any of the AAV serotypes or hybrids or variants thereof.

Viral vectors are obtained by the plasmid co-transfection method. The HEK 293T cells were co-transfected with a helper plasmid containing the AAV2 coat protein gene and a gene that can facilitate AAV replication and a shuttle plasmid pAAV2-CYP4V2 WT or plasmid pAAV2-CYP4V2 opt containing an expression cassette for CYP4V2 gene of interest to form a recombinant adeno-associated virus vector.

### 1.2 Isolation and Purification of Adenoviral Vector

The production of AAV vector uses a three-plasmid system, i.e. a shuttle plasmid containing the target gene CYP4V2, a pRepCap plasmid with the repcap gene of the AAV vector, and a helper plasmid pHelper, and uses PEI as a transfection reagent to co-transfect HEK293 cells and recombine to package an AAV virus vector. Harvesting was performed 48-72 hours after transfection. The harvest solution was purified by affinity chromatography, further purified by anion chromatography, and concentrated by ultrafiltration. The buffer was replaced. The purified recombinant AAV virus vector was subjected to genome titer determination, sterilization, filtering, and dispensing for future use.

### Example 2: In Vitro Expression Assay of Codon-Optimized CYP4V2 Opt

This example evaluated the in vitro expression levels of codon-optimized CYP4V2 opt in two cell lines, HEK293 and ARPE-19. Among them, HEK293 cell line was transfected with plasmid and ARPE-19 cell line was infected with AAV virus to introduce the gene of interest into cells.

Both plasmid AAV2-CYP4V2 WT expressing CYP4V2 WT or plasmid AAV2-CYP4V2 opt expressing CYP4V2 opt were transiently transfected in HEK293 and protein expression in cell lysates was assessed by Western blot analysis (FIG. 2). The expression levels of opt6, opt7, opt8, opt18 (SEQ ID NO: 8, 6, 7, 5) of CYP4V2 gene after codon optimization in HEK293 cells were significantly higher than that of wild-type human CYP4V2 gene (WT).

Protein expression in cell lysates was assessed by Western blot analysis following infection with plasmid AAV2-CYP4V2 WT or plasmid AAV2-CYP4V2 opt in ARPE-19 (FIG. 3).

As can be seen from FIGS. 2 and 3, the codon-optimized CYP4V2 opt gene, in particular opt18, showed significantly higher expression levels compared to the wild-type human CYP4V2 WT gene.

### Example 3: Construction and Characterization of the BCD Cell Model

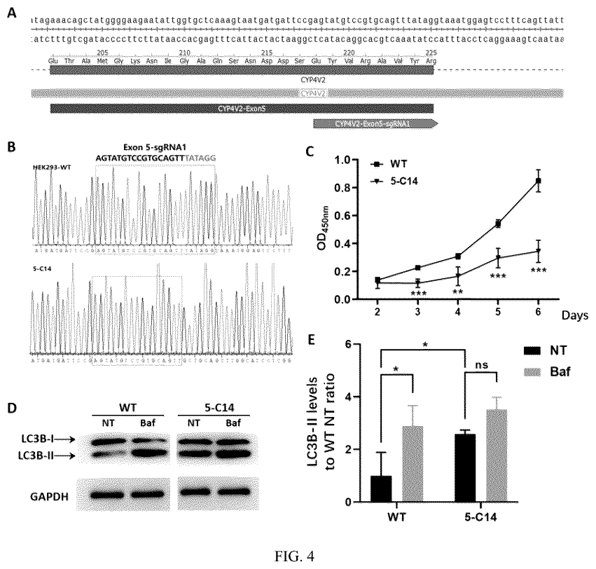

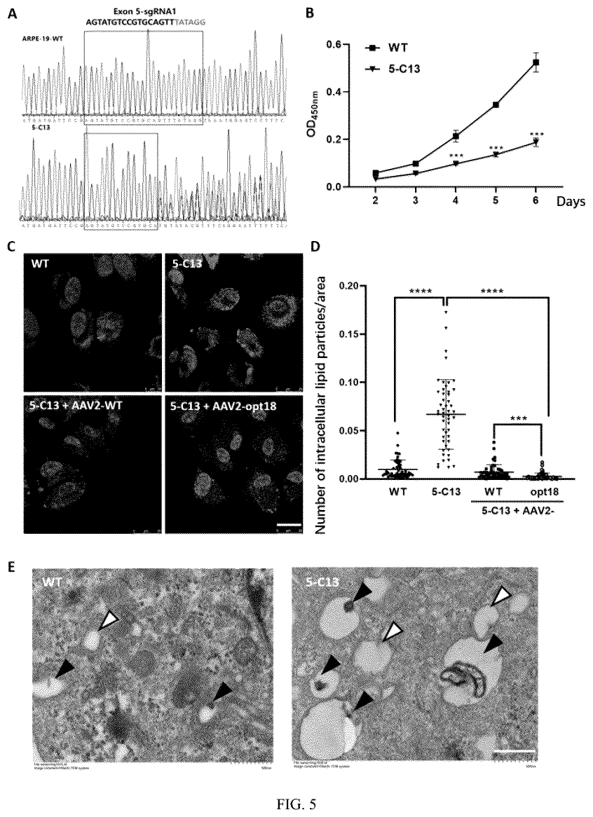

To assess the efficacy of AAV2-CYP4V2 WT and opt genes in BCD gene therapy, this example used the CRISPR-Cas9 method to construct cell models with CYP4V2 mutation in both HEK293 and ARPE-19 cell lines (FIGS. 4 and 5).

The strategy for constructing the cell model is shown in FIG. 4A. Exon5-sgRNA1 (the nucleotide sequence thereof is shown in Table 1) was designed around Exon5; Exon7-sgRNA1, Exon7-sgRNA2, and Exon7-sgRNA3 (the nucleotide sequence thereof is shown in Table 1 respectively) were designed around Exon7; all four sgRNA were constructed into a Cas9-sgRNA vector (Addgene, #58766) to obtain a Cas9-sgRNA plasmid. The Cas9-sgRNA plasmid was transiently transfected into HEK293 and ARPE-19 cells, respectively. 48 H after transfection, puromycin was added to remove the unsuccessfully transfected cells. The cells were then seeded into 96-well culture plates by limiting dilution method and cultured. Two to three weeks later, the genotype of monoclonal cells in the 96-well plate was identified by PCR. The primer sequences used are detailed in Table 1.

TABLE 1
sgRNA and Primer Sequences Used in the
Construction of CYP4V2 Mutant Cells with
CRIPSR/Cas9

| Name | Sequence | Use |
|---|---|---|
| CYP4V2-Exon5- | agtatgtccgtgcagtttatagg | sgRNA vector |
| sgRNA1 | (SEQ ID NO: 18) | construction |
| CYP4V2-Exon7- | catacaggtcatcgctgaacggg | |
| sgRNA1 | (SEQ ID NO: 19) | |
| CYP4V2-Exon7- | tcatacaggtcatcgctgaacgg | |
| sgRNA2 | (SEQ ID NO: 20) | |
| CYP4V2-Exon7- | gattatcattcaaatcatacagg | |
| sgRNA3 | (SEQ ID NO: 21) | |
| CYP4V2-Exon5- | gaaatcacactccaccggga | GenomeDNA |
| F | (SEQ ID NO: 22) | PCR |
| CYP4V2-Exon5- | acctttactgcttaaacacatg | |
| R | ct (SEQ ID NO: 23) | |
| CYP4V2-Exon7- | caggcagcagaaatcgcaag | |
| F | (SEQ ID NO: 24) | |
| CYP4V2-Exon7- | agcctgttcccttcgtcatc | |
| R | (SEQ ID NO: 25) | |
| CYP4V2-ssDNA | taactagggtgcatccaagtcc | Homologous |
| | aaacagaagcatgtgattatca | recombination |
| | ttcaaagcgaacgggccaatga | repair |
| | aatgaacgccaatgaagactgt | template |
| | agaggtgatggcag | |
| | (SEQ ID NO: 26) | |

After screening and amplification of monoclonal cells, cell clones with the correct mutations were verified by gene sequencing, and the results are shown in FIGS. 4B and 5A. The genotypes of the cell clones and the protein product are described in Table 2. The full-length sequence of the wild-type CYP4V2 protein is shown as SEQ ID NO: 27 and its nucleotide sequence as SEQ ID NO: 28. The mutant cells of exon 5 of CYP4V2 had frameshift mutation at 223-224aa of the full-length sequence, and the mutant cells of exon 7 had frameshift mutation at 268-271aa, and expressed a truncated protein compared to the wild type.

TABLE 2
Construction of CYP4V2 mutant cells with CRIPSR/Cas9

| | Clone | | | |
|---|---|---|---|---|
| Mutant | number | Allele | Genome | Predicted protein |
| HEK293-CYP4V2 | Exon | Allele | 668 del T | Frameshift mutation started at 224aa, |
| mutant 1 | 5-C6 | 1/2 | | ended at 226aa |
| HEK293-CYP4V2 | Exon | Allele | 670~673 | Frameshift mutation started at 223aa, |
| mutant 2 | 5-C14 | 1/2 | del 4 ins 67 bp | ended at 237aa |
| HEK293-CYP4V2 | Exon7- | Allele | 814 ins | Frameshift mutation started at 272aa, |
| mutant 3 | C8 | 1/2 | 162 bp | ended at 292aa |
| HEK293-CYP4V2 | Exon7- | Allele | Intron 6 del 8, | 268aa~329aa delete |
| mutant 4 | C30 | 1/2 | 802-810 del 9 | |
| ARPE-19-CYP4V2 | Exon | Allele | 667~674 | Frameshift mutation started at 223aa, |
| mutant 1 | 5-C13 | 1 | del 8 intron | ended at 226aa |
| | | | del 5 | |
| | | Allele | 668 del T | Frameshift mutation started at 224aa, |
| | | 2 | | ended at 226aa |
| ARPE-19-CYP4V2 | Exon | Allele | 668 del T | Frameshift mutation started at 224aa, |
| mutant 2 | 5-C26 | 1 | | ended at 226aa |
| | | Allele | 671~672 | Frameshift mutation started at 224aa, |
| | | 2 | del AT | ended at 224aa |
| ARPE-19-CYP4V2 | Exon | Allele | 668 del T | Frameshift mutation started at 224aa, |
| mutant 3 | 5-C27 | 1 | | ended at 226aa |
| | | Allele | 671~672 | Frameshift mutation started at 224aa, |
| | | 2 | del AT | ended at 224aa |
| ARPE-19-CYP4V2 | Exon | Allele | 810 del T | Frameshift mutation started at 271aa, |
| mutant 4 | 7-C26 | 1 | | ended at 276aa |
| | | Allele | 812~813 | Frameshift mutation started at 271aa, |
| | | 2 | del AA | ended at 274aa |

HEK293 cells having the CYP4V2 mutation were phenotypically characterized by measuring cell proliferation and autophagosome function. It has been reported in the literature that there is a decrease in cell proliferation rate and loss of autophagosome function in RPE cells differentiated from iPSC cells of BCD patients. The present inventors found that the cell proliferation rate of HEK293 mutant cells was significantly reduced compared to HEK293 WT cells. The present inventors evaluated protein levels of the autophagosome marker microtubule-associated protein 1 light chain 3 (LC3) in HEK293 WT and mutant cells. C-terminal processing of LC3 protein can produce LC3-I. Modifications are initiated during autophagosome formation, changing LC3-I to LC3-II. The results showed that the expression level of LC3-II protein was higher in HEK293 mutant cells than in HEK293 WT cells. Bafilomycin-A1, a vacuolar H+-ATPase inhibitor, can increase LC3-II protein levels in WT cells but not in mutant cells. This indicates autophagosome accumulation but impaired autophagy flux in mutant cells. These observations suggest that the CYP4V2 mutation causes a loss of normal cell function, resulting in decreased cell proliferation and defective autophagosome function in HEK293 cells (FIGS. 4C-E). FIG. 4 shows the identification results as represented by 5-C14 cells. Other cell clones had similar phenotypic results.

In the ARPE-19 cell line, which is characterized more closely to RPE cells, the CYP4V2 mutation also causes a change in the cell phenotype. ARPE-19 cells are derived from human retinal pigment epithelial cells that express the surface markers CRALBP and RPE-65 of RPE cells. CYP4V2 mutant ARPE-19 cells were obtained by using CRISPR-Cas9 technology (FIG. 5A). Compared with ARPE-19 WT cells, ARPE-19 mutant cells showed significantly reduced cell proliferation rate (FIG. 5B). CYP4V2 gene encodes hydroxylase involved in fatty acid metabolism in ocular RPE cell layer, and maintains the steady-state balance of retinal polyunsaturated fatty acids. In the CYP4V2 mutant ARPE-19 cell line, fat particle deposition was significantly higher in cells treated with 160 μM arachidonic acid (AA) for 4 consecutive days followed by staining with 5 μM BODIPY 493/503 lipid than in wild-type cells (FIGS. 5C-5D). Transmission electron microscopy revealed a greater number of autophagy vacuoles in the mutant cells (FIG. 5E). These observations suggest that the CYP4V2 mutation causes a loss of normal cell function, resulting in decreased cell proliferation, abnormal fat metabolism, and defective autophagosome function in ARPE-19 cells. FIG. 5 shows the identification results as represented by 5-C13 cells.

### Example 4: In Vitro Pharmacodynamic Evaluation of Codon-Optimized AAV2-CYP4V2 Opt in BCD Cell Model

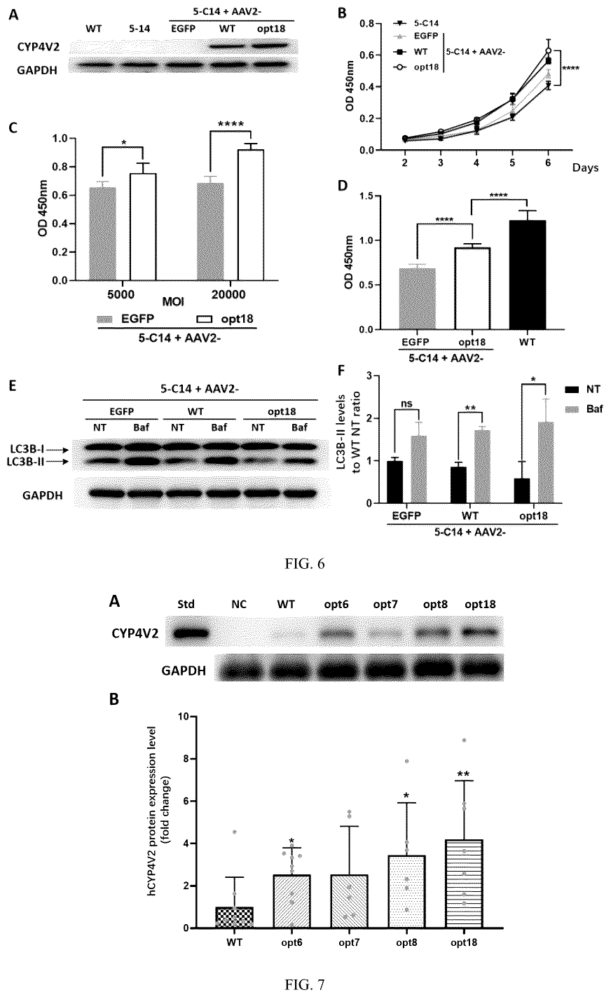

The in vitro potency of codon-optimized CYP4V2 opt was assessed in two BCD cell models, HEK293 and ARPE-19 having the CYP4V2 mutation, following infection with the AAV virus. AAV2 mediated expression of CYP4V2 WT and opt in the HEK293 CYP4V2 mutated BCD cell model is shown in FIG. 6A. The expression of CYP4V2 opt was higher than that of WT under the same amount of virus infection.

Expression of CYP4V2 WT and opt repaired the cell proliferation rate defect of mutant cells in HEK293 BCD cell model in a dose-dependent manner (FIGS. 6B-C). The level of cell proliferation following functional repair of mutant cells by CYP4V2 opt was still lower than that of wild type HEK293 cells, indicating that expression of CYP4V2 opt did not overactivate the cells (FIG. 6D).

Consistent with the decreased proliferation of partially repaired cells, expression of CYP4V2 WT and opt also partially restored the autophagosome function defect of mutant cells. Expression of AAV2-CYP4V2 WT or opt in mutant cells did not significantly repair autophagosome accumulation compared to EGFP controls (without the addition of Baf treatment: NT conditions). However, autophagy flux was significantly increased in mutant cells expressing AAV2-CYP4V2 WT or opt upon treatment with Bafilomycin-Al (Baf), indicating that autophagy function was partially restored (FIGS. 6E-F).

Similarly; AAV2 mediated CYP4V2 WT and opt showed a therapeutic effect in the ARPE-19 BCD cell model with CYP4V2 mutation. ARPE-19 mutant cells 5-C13 were infected by AAV2-WT and AAV2-opt18 viruses, as shown in FIGS. 5C-5D. In ARPE-19 mutant cells 5-C13 infected with AAV2 WT and AAV2 opt18 viruses, compared with uninfected mutant cells, lipid deposition particles were significantly reduced, reaching the level of wild-type ARPE-19 cells. The optimized gene opt18 has a better effect on reducing lipid deposition compared to WT, with significant statistical differences. (FIGS. 5C-5D).

FIGS. 5-6 show the results as represented by CYP4V2 opt18. Similar phenotypic results were observed for the expression of other opt genes.

These results indicate that the phenotypic defect observed in the HEK293 and ARPE-19 BCD cell models is caused by a loss of function of the CYP4V2 gene. Supplementation of the expression of the CYP4V2 gene can restore functional defects in these cells and has the effect of treating BCD.

### Example 5: In Vivo Expression Assay of Codon-Optimized CYP4V2 Opt

Four-week-old wild-type (57BL 6 mice were injected with 1 μl of 1.5×10¹² vg/ml AAV2-CYP4V2 WT and opt recombinant virus particles via subretinal cavity injection. Ocular tissues were sampled every 4 weeks after injection. After homogenizing the mouse eye tissue, RIPA lysate was added to lyse the tissue, and the homogenate was lysed. Equal amounts of total protein were separated by SDS-PAGE, then immunoblotted. CYP4V2 expression levels were detected by Western after protein quantification. Expression levels of the genes were analyzed by Western blot detection. The protein expression level of the CYP4V2 opt gene after codon optimization was compared with WT. The results at 4 weeks post-injection showed that the AAV2-CYP4V2 opt gene was expressed at higher levels in mouse eyes than in WT (FIG. 7).

### Example 6: Phenotypic Characterization of the High Fat-Fed CYP 4V3 KO BCD Mouse Model

The knockout (KO) model of the mouse CYP4V3 gene, which is homologous to the human CYP4V2 gene, has fewer physiological and functional changes compared to BCD patients, and the phenotype appears much later than in patients. Six-month-old mice (equivalent to 20-30 years of age in humans) had sporadic ocular lipid deposits, and 12-month-old mice (equivalent to 50 years of age in humans, already suffering from ocular blindness) had significant lipid deposits without ocular ERG function and loss of vision (Lockhart, C. M., et al. (2014). “Generation and characterization of a murine model of Bietti crystalline dystrophy.” Invest Ophthalmol Vis Sci 55 (9): 5572-5581). The occurrence of retinopathy was accelerated and aggregated in the CYP4V3 KO mouse model after administration of a high-fat diet (HFD) (Qu, B., et al. (2020). “Treating Bietti crystalline dystrophy in a high-fat diet-exacerbated murine model using gene therapy.” Gene Ther 27 (7-8): 370-382). In this example, HFD feeding was performed after birth and weaning. Fundus imaging, OCT, and ERG functional tests were performed every 4 weeks after 4 weeks of age.

### Example 7: In Vivo Pharmacodynamic Evaluation of Codon-Optimized AAV2-CYP4V2 Opt in BCD Mouse Model

1 μl of 5e¹² vg/ml AAV2-GFP or CYP4V2 opt virus particles were injected in the eyeballs of high-fat fed CYP4V3 KO BCD mice with pathological changes of the ocular fundus using subretinal space injection. OCT and ERG functional tests were performed every 4 weeks after injection, and the therapeutic effects of AAV2-GFP and CYP4V2 opt virus particles were compared and analyzed.

1.-10. (canceled)

11. A polynucleotide encoding a CYP4V2 protein, with the nucleotide sequence as shown in SEQ ID NO: 5.

12. An expression cassette comprising the polynucleotide of claim 11 and a promoter operably linked to the polynucleotide.

13. An expression vector comprising the polynucleotide of claim 11 or an expression cassette comprising the polynucleotide.

14. The expression vector of claim 13, further comprising an expression control element to which the polynucleotide encoding the CYP4V2 protein is operably linked.

15. The expression vector of claim 14, wherein the expression control element is selected from one or more of an origin of replication, a promoter, an enhancer, an intron, a polyA signal, ITR, an insulator.

16. The expression vector of claim 13, wherein the expression vector further comprises an origin of replication.

17. The expression vector of claim 16, wherein the origin of replication sequence is selected from f1 bacteriophage ori, RK2oriV, pUC ori or pSC101ori.

18. The expression vector of claim 13, wherein the expression vector further comprises a 5′ ITR.

19. The expression vector of claim 18, wherein the nucleotide sequence of the 5′ ITR is as shown in SEQ ID NO: 1.

20. The expression vector of claim 13, wherein the expression vector further comprises a 3′ ITR.

21. The expression vector of claim 20, wherein the nucleotide sequence of the 3′ ITR is as shown in SEQ ID NO: 11.

22. The expression vector of claim 13, wherein the expression vector further comprises an enhancer.

23. The expression vector of claim 22, wherein the enhancer is a CMV enhancer.

24. The expression vector of claim 23, wherein the nucleotide sequence of the enhancer is as shown in SEQ ID NO: 2.

25. The expression vector of claim 13, wherein the expression vector further comprises a promoter.

26. The expression vector of claim 25, wherein the promoter is a specific or non-specific promoter.

27. The expression vector of claim 26, wherein the promoter is selected from a CBA promoter, CMV promoter, SV40 promoter, hPGK promoter and TRE3GS promoter.

28. The expression vector of claim 27, wherein the promoter is a CBA promoter.

29. The expression vector of claim 28, wherein the nucleotide sequence of the CBA promoter is as shown in SEQ ID NO: 3.

30. The expression vector of claim 25, wherein the promoter is an inducible promoter.

31. The expression vector of claim 30, wherein the inducible promoter comprises one or more of a tetracycline-regulated promoter, an alcohol-regulated promoter, a steroid-regulated promoter, a metal-regulated promoter, a pathogen-regulated promoter, a temperature/heat-inducible promoter, a light-regulated promoter, and a IPTG-inducible promoter.

32. The expression vector of claim 31, wherein the tetracycline-regulated promoter is selected from a Tet on promoter, a Tet off promoter, or a Tet Activator promoter.

33. The expression vector of claim 31, wherein the alcohol-regulated promoter is selected from an alcohol dehydrogenase I (alcA) gene promoter, a promoter responsive to an alcohol transactivator protein (AlcR).

34. The expression vector of claim 31, wherein the steroid-regulated promoter is selected from a rat glucocorticoid receptor promoter, a human estrogen receptor promoter, a moth ecdysone receptor promoter, a retinoid promoter, or a thyroid receptor superfamily promoter.

35. The expression vector of claim 31, wherein the metal-regulated promoter is selected from yeast, mouse or human metallothionein promoters.

36. The expression vector of claim 31, wherein the pathogenic-regulated promoter is selected from a salicylic acid-regulated promoter, an ethylene-regulated promoter or a BTH promoter.

37. The expression vector of claim 31, wherein the temperature/heat inducible promoter is selected from an HSP-70 promoter, an HSP-90 promoter, or a soybean heat shock promoter.

38. The expression vector of claim 31, wherein the light-regulated promoter is a light-responsive promoter of a plant cell.

39. The expression vector of claim 13, wherein the expression vector further comprises an exon and an intron.

40. The expression vector of claim 39, wherein the exon and intron are the first exon and first intron of the chicken β-actin gene.

41. The expression vector of claim 40, wherein the nucleotide sequence of the exon and intron is as shown in SEQ ID NO: 4.

42. The expression vector of claim 13, wherein the expression vector further comprises a polyA signal.

43. The expression vector of claim 42, wherein the polyA signal is bovine growth hormone poly A, SV40 polyA, or human beta globin poly A.

44. The expression vector of claim 43, wherein the nucleotide sequence of the polyA signal is as shown in SEQ ID NO: 10.

45. The expression vector of claim 13, wherein the nucleotide sequence of the expression vector is as shown in SEQ ID NO: 12, SEQ ID NO: 13, SEQ ID NO: 14, SEQ ID NO: 15, or SEQ ID NO: 16.

46. The expression vector of claim 13, wherein the expression vector further comprises a post-transcriptional regulatory element.

47. The expression vector of claim 46, wherein the post-transcriptional regulatory element is a woodchuck hepatitis virus post-transcriptional regulatory element.

48. The expression vector of claim 13, wherein the expression vector further comprises a gene encoding a marker.

49. The expression vector of claim 48, wherein the marker is selected from one or more of an antibiotic resistance protein, a toxin resistance protein, a colored or fluorescent protein.

50. The expression vector of claim 49, wherein the antibiotic is selected from ampicillin, neomycin, G418, puromycin and blasticidin.

51. The expression vector of claim 49, wherein the toxin is selected from anthrax toxin and diphtheria toxin.

52. The expression vector of claim 49, wherein the colored or fluorescent protein is selected from a green fluorescent protein, an enhanced green fluorescent protein, a red fluorescent protein, and luciferase.

53. The expression vector of claim 48, wherein the marker is dihydrofolate reductase.

54. The expression vector of claim 14, wherein the expression vector is a plasmid.

55. The expression vector of claim 14, wherein the expression vector is a cosmid.

56. The expression vector of claim 14, wherein the expression vector is a viral vector.

57. The expression vector of claim 14, wherein the expression vector is an RNA vector.

58. The expression vector of claim 14, wherein the expression vector is a linear or circular DNA or RNA molecule.

59. The expression vector of claim 54, wherein the plasmid is selected from pCI, puc57, pcDNA3, pSG5, pJ603, and pCMV.

60. The expression vector of claim 56, wherein the viral vector is selected from retrovirus, adeno-associated virus, coronavirus, influenza virus, rabies virus, vesicular stomatitis virus, measles virus, Sendai virus, alphaviruses, herpes simplex virus types 1 and 2, Epstein-Bar virus, cytomegalovirus, cowpox virus, fowlpox virus, and canarypox virus, norwalk virus, togavirus, flavivirus, reovirus, papovavirus, hepadnavirus, baculovirus, and hepatitis virus.

61. The expression vector of claim 56, wherein the viral vector is a picornavirus.

62. The expression vector of claim 60, the retrovirus is selected from mammalian C-type, lentivirus and spumavirus.

63. The expression vector of claim 62, wherein the lentiviral vector is selected from HIV-1, HIV-2, SIV, FIV, BIV, EIAV, CAEV and ovine demyelinating leukoencephalitis lentivirus.

64. The expression vector of claim 60, wherein the expression vector is an adeno-associated virus.

65. The expression vector of claim 64, wherein the adeno-associated virus is selected from AAV type 1, AAV type 2, AAV type 3, AAV type 4, AAV type 5, AAV type 6, AAV type 7, AAV type 8, AAV type 9, AAV type 10.

66. The expression vector of claim 13, wherein the expression vector further comprises a shortened chimeric intron and a Kozak initiation sequence.

67. The expression vector of claim 66, wherein the shortened chimeric intron is as shown in SEQ ID NO: 4.

68. The expression vector of claim 66, wherein the nucleotide sequence of the Kozak initiation sequence is as shown in SEQ ID NO: 17.

69. A viral particle comprising the polynucleotide of claim 11.

70. A viral particle comprising the expression cassette of claim 12.

71. A viral particle comprising the expression vector of any claim 13.

72. A pharmaceutical composition for the treatment of BCD comprising the polynucleotide of claim 11 and a pharmaceutically acceptable carrier, wherein the pharmaceutical composition expresses wild type or codon-optimized CYP4V2 protein.

73. Use of the polynucleotide of claim 11 for the preparation of a medicament for the treatment of Bietti crystalline dystrophy.

74. Use of the pharmaceutical composition of claim 72 for the preparation of a medicament for the treatment of Bietti crystalline dystrophy.
